(19) United States
(12) Patent Application Publication
(10) Pub. No.: US 2024/0240047 A1
(43) Pub. Date: Jul. 18, 2024

(54) BIOMEDICAL DEVICES HAVING A SURFACE COATING

(71) Applicant: Bausch + Lomb Ireland Limited, Dublin (IE)

(72) Inventors: Mohammad Vatankhah Varnosfaderani, Rochester, NY (US); Mark R. Mis, Rush, NY (US); Feng-Yang Shih, Rochester, NY (US); Jade J. Russell, Perry, NY (US); Alok Kumar Awasthi, Pittsford, NY (US)

(21) Appl. No.: 18/540,063

(22) Filed: Dec. 14, 2023

Related U.S. Application Data
(60) Provisional application No. 63/436,976, filed on Jan. 4, 2023.

(51) Int. Cl. C09D 153/00; C08F 293/00; G02C 7/04

(52) U.S. Cl. CPC C09D 153/00; C08F 293/005; G02C 7/049; C08F 2438/01

(57) ABSTRACT

A biomedical device having a surface coating includes a bulk material having one or more biomedical device surface reactive functional groups and/or one or more biomedical device surface protected reactive functional groups and a surface coating. The surface coating is derived from a block copolymer including (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

### PRIORITY CLAIM

The present application claims priority to U.S. Provisional Patent Application Ser. No. 63/436,976, entitled “Biomedical Devices Having a Surface Coating,” filed Jan. 4, 2023, the content of which is incorporated by reference herein in its entirety.

### BACKGROUND

Biomedical devices such as ophthalmic lenses made from, for example, silicone-containing materials, have been investigated for a number of years. Such materials can generally be subdivided into two major classes, namely hydrogels and non-hydrogels. Hydrogels can absorb and retain water in an equilibrium state, whereas non-hydrogels do not absorb appreciable amounts of water. Regardless of their water content, both hydrogel and non-hydrogel silicone medical devices tend to have relatively hydrophobic, non-wettable surfaces that have a high affinity for lipids. This problem is of particular concern with contact lenses.

Those skilled in the art have long recognized the need for modifying the surface of the biomedical devices such as silicone contact lenses so that they are compatible with the eye. For example, by increasing the hydrophilicity of a contact lens surface, the wettability of the contact lens can be improved. This, in turn, is associated with improved wear comfort of the contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

### SUMMARY

In accordance with an illustrative embodiment, a biomedical device having a surface coating comprises a bulk material having one or more biomedical device surface reactive functional groups and/or one or more biomedical device surface protected reactive functional groups and a surface coating, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

In accordance with another illustrative embodiment, a method for making a biomedical device having a surface coating comprises forming a surface coating on a biomedical device having one or more biomedical device surface reactive functional groups and/or one or more biomedical device surface protected reactive functional groups, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

### BRIEF DESCRIPTION OF THE DRAWINGS

Exemplary embodiments will be described below in more detail, with reference to the accompanying drawings, of which:

FIG. 1 shows the results of the co-efficient of friction for Example 6 and Comparative Example 1.

FIGS. 2A and 2B show the XPS results for Examples 10 and 11 and Comparative Example 3.

### DETAILED DESCRIPTION

Various illustrative embodiments described herein include surface modified biomedical devices. For example, by increasing the hydrophilicity of a biomedical device such as a contact lens surface, the wettability of the contact lens can be improved. This, in turn, is associated with improved wear comfort of the contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

Accordingly, it would be desirable to provide improved biomedical devices having a highly wettable and/or lubricious surface coating on the biomedical device as compared to a non-treated biomedical device such that the biomedical device has an optically clear, hydrophilic surface coating that will not only exhibit improved wettability, but which may generally allow the use of a contact lens in the human eye for an extended period of time. In addition, the illustrative embodiments described herein provide a biomedical device with an optically clear, hydrophilic surface coating prepared in a simple and cost-efficient manner.

The non-limiting illustrative embodiments disclosed herein are directed to a biomedical device having a surface coating comprising a bulk material having one or more biomedical device surface reactive functional groups and/or one or more biomedical device surface protected reactive functional groups and a surface coating, the surface coating being derived from a block copolymer comprising monomeric units derived from an ethylenically unsaturated containing monomer having reactive functionalities or nucleofugic functionalities that are complementary to the one or more biomedical device surface reactive functional groups and the one or more biomedical device surface protected reactive functional groups, and monomeric units derived from an ethylenically unsaturated-containing hydrophilic monomer.

As used herein, a “biomedical device” is any article that is designed to be used while either in or on mammalian tissues or fluid, and preferably in or on human tissue or fluids. Representative examples of biomedical devices include, but are not limited to, artificial ureters, diaphragms, intrauterine devices, heart valves, catheters, denture liners, prosthetic devices, ophthalmic lens applications, where the lens is intended for direct placement in or on the eye, such as, for example, intraocular devices and contact lenses. In an illustrative embodiment, a biomedical device is an ophthalmic device. In another illustrative embodiment, a biomedical device is a contact lens. In yet another illustrative embodiment, a biomedical device is a silicone hydrogel.

As used herein, the term “ophthalmic device” refers to ophthalmic devices that reside in or on the eye. These devices can provide optical correction, wound care, drug delivery, diagnostic functionality or cosmetic enhancement or effect or a combination of these properties. Suitable ophthalmic devices include, for example, ophthalmic lenses such as soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking.

The biomedical devices to be surface modified according to the non-limiting illustrative embodiments disclosed herein can be any material known in the art capable of forming a biomedical device as described above. In one embodiment, biomedical devices include devices which are formed from materials not hydrophilic per se. Such devices are formed from materials known in the art and include, by way of example, polysiloxanes, perfluoropolyethers, fluorinated poly(meth)acrylates or equivalent fluorinated polymers derived, e.g., from other polymerizable carboxylic acids, polyalkyl (meth)acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol. Representative examples of suitable bulk materials include, but are not limited to, Lotrafilcon A, Neofocon, Pasifocon, Telefocon, Silafocon, Fluorsilfocon, Paflufocon, Silafocon, Elastofilcon, Fluorofocon or Teflon AF materials, such as Teflon AF 1600 or Teflon AF 2400 which are copolymers of about 63 to about 73 mol % of perfluoro-2,2-dimethyl-1,3-dioxol and about 37 to about 27 mol % of tetrafluoroethylene, or of about 80 to about 90 mol % of perfluoro-2,2-dimethyl-1,3-dioxol and about 20 to about 10 mol % of tetrafluoroethylene.

In another embodiment, biomedical devices include devices which are formed from materials hydrophilic per se, since reactive groups, e.g., carboxy, carbamoyl, sulfate, sulfonate, phosphate, amine, ammonium or hydroxy groups, are inherently present in the material and therefore also at the surface of a biomedical device manufactured therefrom. Such devices are formed from materials known in the art and include, by way of example, polyhydroxyethyl acrylate, hydroxyethyl methacrylate (HEMA), polyvinyl pyrrolidone (PVP), polyacrylic acid, polymethacrylic acid, polyacrylamide, polydimethylacrylamide (DMA), polyvinyl alcohol and the like and copolymers thereof, e.g., from two or more monomers selected from hydroxyethyl acrylate, hydroxyethyl methacrylate, N-vinyl pyrrolidone, acrylic acid, methacrylic acid, acrylamide, dimethyl acrylamide, vinyl alcohol and the like. Representative examples of suitable bulk materials include, but are not limited to, Polymacon, Tefilcon, Methafilcon, Deltafilcon, Bufilcon, Phemfilcon, Ocufilcon, Focofilcon, Etafilcon, Hefilcon, Vifilcon, Tetrafilcon, Perfilcon, Droxifilcon, Dimefilcon, Isofilcon, Mafilcon, Nelfilcon, Atlafilcon and the like.

In another embodiment, biomedical devices include devices which are formed from materials which are amphiphilic segmented copolymers containing at least one hydrophobic segment and at least one hydrophilic segment which are linked through a bond or a bridge member.

It is particularly useful to employ biocompatible materials herein including both soft and rigid materials commonly used for ophthalmic lenses, including contact lenses. In general, non-hydrogel materials are hydrophobic polymeric materials that do not contain water in their equilibrium state. Typical non-hydrogel materials comprise silicone acrylics, such as those formed bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups (polysiloxanes are also commonly known as silicone polymers).

On the other hand, hydrogel materials comprise hydrated, cross-linked polymeric systems containing water in an equilibrium state. Hydrogel materials contain about 5 weight percent water or more (up to, for example, about 80 weight percent). In one embodiment, hydrogel materials, include silicone hydrogel materials. In another embodiment, hydrogel materials include vinyl functionalized polydimethylsiloxanes copolymerized with hydrophilic monomers as well as fluorinated methacrylates and methacrylate functionalized fluorinated polyethylene oxides copolymerized with hydrophilic monomers. Representative examples of suitable hydrogel materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

In one embodiment, hydrogel materials for biomedical devices, such as contact lenses, can contain a hydrophilic monomer such as one or more unsaturated carboxylic acids, vinyl lactams, amides, polymerizable amines, vinyl carbonates, vinyl carbamates, oxazolone monomers, copolymers thereof and the like and mixtures thereof. Useful amides include acrylamides such as N,N-dimethylacrylamide and N,N-dimethylmethacrylamide. Useful vinyl lactams include cyclic lactams such as N-vinyl-2-pyrrolidone. Examples of other hydrophilic monomers include hydrophilic prepolymers such as poly(alkene glycols) functionalized with polymerizable groups. Examples of useful functionalized poly(alkene glycols) include poly(diethylene glycols) of varying chain length containing monomethacrylate or dimethacrylate end caps. In some embodiments, the poly(alkene glycol) polymer contains at least two alkene glycol monomeric units. S till further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. In another embodiment, a hydrogel material can contain a siloxane-containing monomer and at least one of the aforementioned hydrophilic monomers and/or prepolymers.

In some embodiments, hydrogel materials may further include one or more hydrophobic monomers. Suitable hydrophobic monomers include, for example, substitute or unsubstituted C₁ to C₂₀ alkyl and C₃ to C₂₀ cycloalkyl (meth)acrylates such as (2-amino)ethyl methacrylate and methacrylic acid, substituted and unsubstituted aryl (meth)acrylates (wherein the aryl group comprises 6 to 36 carbon atoms), (meth) acrylonitrile, styrene, lower alkyl styrene, lower alky vinyl ethers, and C₂ to C₁₀ perfluroalkyl (meth)acrylates and correspondingly partially fluorinate (meth)acrylates.

In addition, a wide variety of materials can be used herein, and silicone hydrogel contact lens materials are particularly preferred. Silicone hydrogels generally have a water content greater than about 5 weight percent and more commonly between about 10 to about 80 weight percent. Such materials are usually prepared by polymerizing a mixture containing at least one silicone-containing monomer and at least one hydrophilic monomer. Typically, either the silicone-containing monomer or the hydrophilic monomer functions as a crosslinking agent (a crosslinker being defined as a monomer having multiple polymerizable functionalities) or a separate crosslinker may be employed. Applicable silicone-containing monomers for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

In an illustrative embodiment, as may be combined with one or more of the preceding paragraphs, the one or more silicone-containing monomers can include, as a class of representative silicone-containing monomers, one or more bulky silicon-containing monomers. In an illustrative embodiment, an example of one or more bulky silicon-containing monomers includes bulky polysiloxanylalkyl(meth)acrylic monomers represented by the structure of Formula

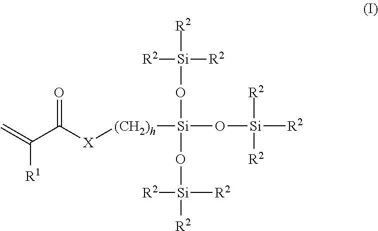

wherein X denotes —O— or —NR— wherein R denotes hydrogen or a C₁-C₄ alkyl; each R¹ independently denotes hydrogen or methyl; each R² independently denotes a lower alkyl radical, a phenyl radical or a group represented by

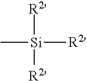

wherein each R²′ independently denotes a lower alkyl or phenyl radical; and h is 1 to 10.

In an illustrative embodiment, an example of one or more bulky silicon-containing monomers includes bulky polysiloxanylalkyl carbamate monomers represented by the structure of Formula Ia:

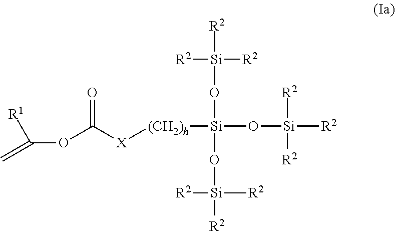

wherein X denotes —NR—; wherein R denotes hydrogen or a C₁-C₄ alkyl; R¹ denotes hydrogen or methyl; each R² independently denotes a lower alkyl radical, phenyl radical or a group represented by

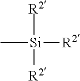

wherein each R²′ independently denotes a lower alkyl or phenyl radical; and h is 1 to 10, and the like.

Examples of bulky monomers include 3-methacryloyloxypropyltris(trimethyl-siloxy)silane or tris(trimethylsiloxy)silylpropyl methacrylate, sometimes referred to as TRIS and tris(trimethylsiloxy)silylpropyl vinyl carbamate, sometimes referred to as TRIS-VC and the like and mixtures thereof.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

In an illustrative embodiment, as may be combined with one or more of the preceding paragraphs, the one or more silicone-containing monomers can include, as a class of representative silicone-containing monomers, one or more silicone-containing vinyl carbonate or vinyl carbamate monomers. Suitable silicone-containing vinyl carbonate or vinyl carbamate monomers include, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

In an illustrative embodiment, as may be combined with one or more of the preceding paragraphs, the one or more silicone-containing monomers can include, as a class of representative silicone-containing monomers, one or more polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels”, Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae II and III:

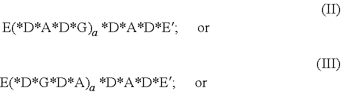

wherein:
D independently denotes an alkyl diradical, an alkyl cycloalkyl diradical, a cycloalkyl diradical, an aryl diradical or an alkylaryl diradical having 6 to about 30 carbon atoms;
G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
* denotes a urethane or ureido linkage;
a is at least 1;

A independently denotes a divalent polymeric radical of Formula IV:

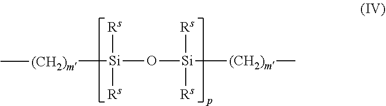

wherein each R$ independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;

each of E and E′ independently denotes a polymerizable unsaturated organic radical represented by Formula V:

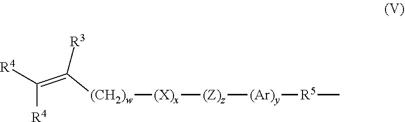

wherein: R³ is hydrogen or methyl;
R⁴ is hydrogen, an alkyl radical having 1 to 6 carbon atoms, or a —CO—Y—R⁶ radical wherein
Y is —O—, —S— or —NH—;
R⁵ is a divalent alkylene radical having 1 to about 10 carbon atoms;
R⁶ is a alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

In some embodiments, a silicone-containing urethane monomer can be represented by Formula VI:

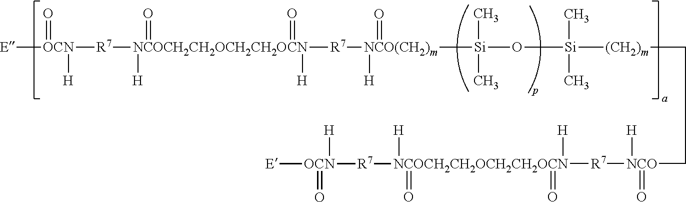

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

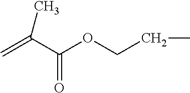

In another illustrative embodiment, a silicone hydrogel material comprises (in bulk, that is, in the monomer mixture that is copolymerized) about 5 to about 50 percent, and preferably about 10 to about 25, by weight of one or more silicone macromonomers, about 5 to about 75 percent, and preferably about 30 to about 60 percent, by weight of one or more polysiloxanylalkyl (meth)acrylic monomers, and about 10 to about 50 percent, and preferably about 20 to about 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the illustrative embodiments. The silane macromonomer may be a silicon-containing vinyl carbonate or vinyl carbamate or a polyurethane-polysiloxane having one or more hard-soft-hard blocks and end-capped with a hydrophilic monomer.

In an illustrative embodiment, as may be combined with one or more of the preceding paragraphs, the one or more silicone-containing monomers can include, as a class of representative silicone-containing monomers, one or more fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF2)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as substrates that can benefit by being coated with the block copolymer disclosed herein and have been disclosed in various publications and are being continuously developed for use in contact lenses and other medical devices can also be used. For example, a biomedical device can be formed from at least a cationic monomer such as cationic silicone-containing monomer or cationic fluorinated silicone-containing monomers.

As one skilled in the art will readily appreciate, the one or more biomedical device surface reactive functional groups of the biomedical device disclosed herein may be inherently present at the surface of the biomedical device. However, if the biomedical device contains too few or no functional groups, the surface of the biomedical device can be modified by known techniques, for example, plasma chemical methods or conventional functionalization with groups such as —OH, —NH₂ or —CO₂H. For example, the surface of the biomedical device can be treated with a plasma discharge or corona discharge to introduce or increase the population of biomedical device surface functional groups. The type of gas introduced into the treatment chamber will depend on the desired type of biomedical device surface functional group. For example, hydroxyl surface groups can be produced with a treatment chamber atmosphere containing water vapor or alcohols. Carboxyl surface groups can be produced with a treatment chamber atmosphere containing oxygen, air or another oxygen-containing gas. Amino surface groups can be produced with a treatment chamber atmosphere containing ammonia or an amine source. Mercaptan surface groups can be produced with a treatment chamber atmosphere containing sulfur-containing gases such as organic mercaptans or hydrogen sulfide. As one skilled in the art will readily appreciate, a combination of any of the foregoing gases can be used in the treatment chamber to produce a combination of biomedical device surface functional groups on the surface of the biomedical device. Methods and apparatus for surface treatment by plasma discharge are disclosed in, for example, U.S. Pat. Nos. 6,550,915 and 6,794,456, the contents of which are incorporated by reference herein.

Suitable biomedical device surface functional groups of the biomedical devices disclosed herein include a wide variety of groups well known to the skilled artisan. Representative examples of such functional groups include, but are not limited to, a hydroxy group, a tosylate group, a mesylate group, a triflate group, a nosyloxy group, an amino group, a carboxy group, a carbonyl group, an aldehyde group, a sulfonic acid group, a sulfonyl chloride group, an isocyanato group, a carboxy anhydride group, a lactone group, an azlactone group, an epoxy group, a group being replaceable by amino or hydroxy groups, such as halo groups, a group capable of undergoing Michael addition-type reaction and mixtures thereof. In one embodiment, the biomedical device surface functional groups of the biomedical device are amino groups and/or hydroxy groups.

In one or more additional non-limiting illustrative embodiments, as may be combined with one or more of the preceding paragraphs, the biomedical device disclosed herein can have one or more biomedical device surface protected reactive functional groups. The one or more biomedical device surface protected reactive functional groups can be attached to one or more of the monomers utilized in forming the biomedical device. The one or more biomedical device surface protected functional groups of the monomers utilized in forming the biomedical device are used when one or more of the groups of the monomers utilized in forming the biomedical device may be incompatible with the polymerization process, incompatible with other monomers in the polymerization process, or to further functionalize the biomedical device post polymerization.

In order for these protected monomers to subsequently react with a ring opening functionality of a coating block polymer they must be deprotected prior to exposure to the block polymer. The deprotection of the monomers is accomplished by methods that are widely known by those skilled in the art. In an illustrative embodiment, an example includes the deprotection of a BOC-protected amine by simple carbamate hydrolysis in acidic conditions. For example, the starting material is dissolved in water or an organic solvent, and then treated with a concentrated hydrochloric acid, or trifluoroacetic acid (TFA) which are typically the utilized acids. In another illustrative embodiment, an additional example includes the hydrolysis of protected acids by catalytic amounts of acids or bases under heat. For example, the TMS group in 2-(Trimethylsilyloxy)ethyl methacrylate is extremely labile to acid- or base-catalyzed solvolysis, to attack by many nucleophiles, and also to hydrogenolysis.

In non-limiting illustrative embodiments, the one or more protected monomers can include, for example, a protected HEMA, protected (2-amino)ethyl methacrylate, protected methacrylic acid and the like. These protected monomers are merely exemplary and should not be construed as limiting the protected monomers. All other protected monomers are contemplated herein. In illustrative embodiments, the following are representative examples of protected monomers.

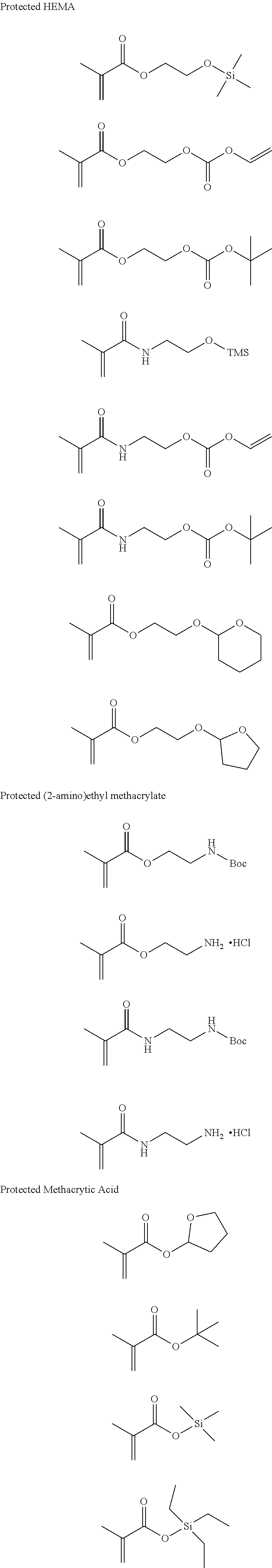

The protected monomers are either commercially available from such sources as Polysciences, Sigma-Aldrich, TCI, Flourochem, Wako, and Sigma-Aldrich, or can be prepared according to methods known in the art. For example, the following protected monomers can be prepared by the following Schemes I to X below.

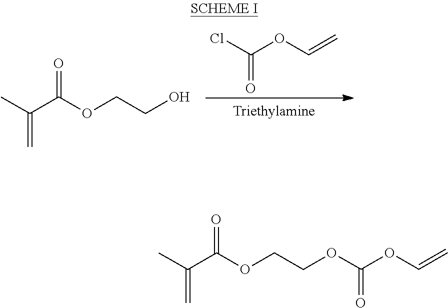

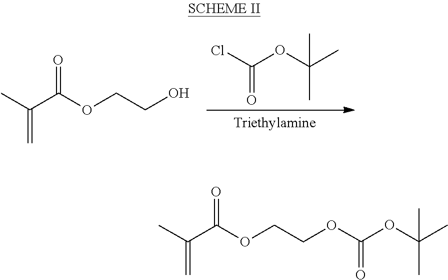

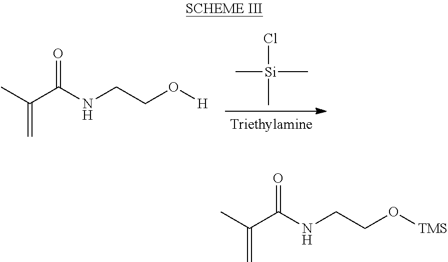

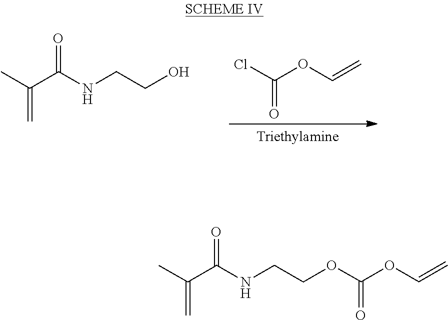

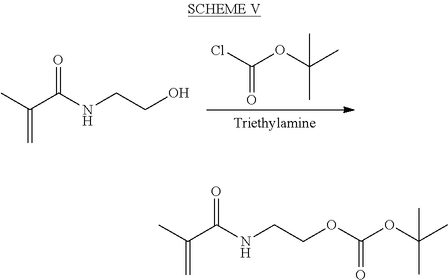

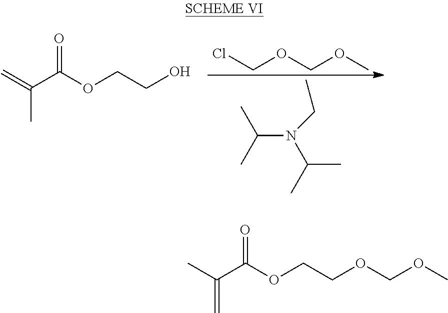

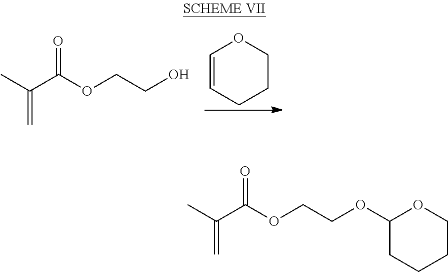

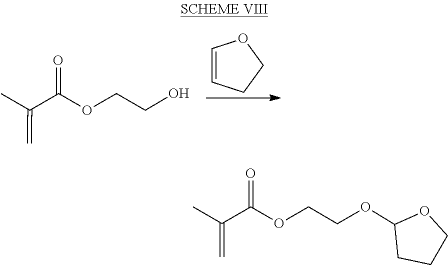

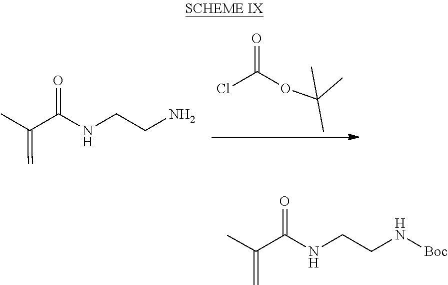

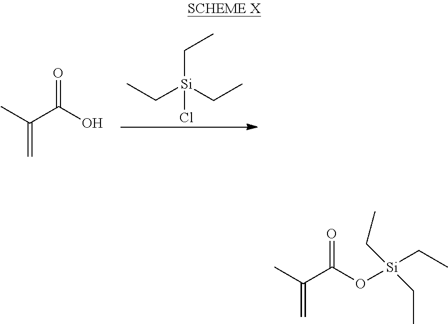

According to non-limiting illustrative embodiments disclosed herein, the foregoing biomedical device having one or more biomedical device surface reactive functional groups and/or one or more biomedical device surface protected reactive functional groups are then exposed to a block copolymer comprising (a) monomeric units derived from an ethylenically unsaturated containing monomer having ring-opening reactive functionalities that are complementary to the biomedical device surface reactive functional groups and (b) monomeric units derived from an ethylenically unsaturated-containing hydrophilic monomer to covalently attach the ring-opening reactive functionalities of the monomeric units derived from an ethylenically unsaturated containing monomer having ring-opening reactive functionalities to the surface of the biomedical device through the biomedical device surface reactive functional groups. In an illustrative embodiment, the block copolymer is a biomedical device surface coating-forming block copolymer. In an illustrative embodiment, the block copolymer is a brush copolymer. The term “polymer brushes,” as used herein is understood to mean a polymer brush that contains polymer chains, one end of which is directly or indirectly tethered to a surface and another end of which is free to extend from the surface, somewhat analogous to the bristles of a brush. In another illustrative embodiment, the block copolymer is a comb-like copolymer.

Representative examples of the ethylenically unsaturated moiety of the ethylenically unsaturated containing monomer having ring-opening reactive functionalities and the ethylenically unsaturated-containing hydrophilic monomer include, by way of example, (meth)acrylate-containing radicals, (meth)acrylamido-containing radicals, vinylcarbonate-containing radicals, vinylcarbamate-containing radicals, styrene-containing radicals, itaconate-containing radicals, vinyl-containing radicals, vinyloxy-containing radicals, fumarate-containing radicals, maleimide-containing radicals, vinylsulfonyl radicals and the like. As used herein, the term “(meth)” denotes an optional methyl substituent. Thus, for example, terms such as “(meth)acrylate” denotes either methacrylate or acrylate, and “(meth)acrylamide” denotes either methacrylamide or acrylamide.

In one embodiment, an ethylenically unsaturated moiety is represented by the general formula:

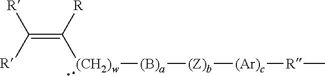

wherein R is hydrogen or a alkyl group having 1 to 6 carbon atoms such as methyl; each R′ is independently hydrogen, an alkyl radical having 1 to 6 carbon atoms, or a —CO—Y—R″′ radical wherein Y is —O—, —S— or —NH— and R″′ is an alkyl radical having 1 to about 10 carbon atoms; R″ is a linking group (e.g., a divalent alkenyl radical having 1 to about 12 carbon atoms); B denotes —O— or —NH—; Z denotes —CO—, —OCO— or —COO—; Ar denotes an aromatic radical having 6 to about 30 carbon atoms; w is 0 to 6; a is 0 or 1; b is 0 or 1; and c is 0 or 1. The ethylenically unsaturated-containing moiety can be attached to the ethylenically unsaturated containing monomer having ring-opening reactive functionalities and the ethylenically unsaturated-containing hydrophilic monomer as pendent groups, terminal groups or both.

In an illustrative embodiment, ethylenically unsaturated containing monomers having ring-opening reactive functionalities that are complementary to the biomedical device surface reactive functional groups include ethylenically unsaturated epoxy-containing monomers. Suitable ethylenically unsaturated epoxy-containing monomers include, for example, glycidyl-containing ethylenically unsaturated monomers such as glycidyl methacrylate, glycidyl acrylate, glycidyl vinylcarbonate, glycidyl vinylcarbamate, vinylcyclohexyl-1,2-epoxide and the like.

In another illustrative embodiment, ethylenically unsaturated containing monomers having ring-opening reactive functionalities that are complementary to the biomedical device surface reactive functional groups include ring-opening reactive monomers having an azlactone group represented by the following formula:

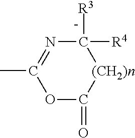

wherein R³ and R⁴ are independently an alkyl group having 1 to 14 carbon atoms, a cycloalkyl group having 3 to about 14 carbon atoms, an aryl group having 5 to about 12 ring atoms, an arenyl group having 6 to about 26 carbon atoms, and 0 to 3 heteroatoms selected from S, N, and O, or R³ and R⁴ taken together with the carbon to which they are joined can form a carbocyclic ring containing 4 to 12 ring atoms, and n is an integer 0 or 1. Such monomeric units are disclosed in, for example, U.S. Pat. No. 5,177,165.

The ring structure of such reactive functionalities is susceptible to nucleophilic ring-opening reactions with complementary reactive functional groups on the surface of substrate being treated. For example, the azlactone functionality can react with primary amines, hydroxyl radicals or the like which may be present on the surface of the device to form a covalent bond between the substrate and the hydrophilic reactive polymer at one or more locations along the polymer. A plurality of attachments can form a series of polymer loops on the substrate, wherein each loop comprises a hydrophilic chain attached at both ends to the substrate.

Azlactone-functional monomers for making the block copolymer can be any monomer, prepolymer, or oligomer comprising an azlactone functionality of the above formula in combination with a vinylic group on an unsaturated hydrocarbon to which the azlactone is attached. In one embodiment, azlactone-functionality is provided in the hydrophilic polymer by 2-alkenyl azlactone monomers. The 2-alkenyl azlactone monomers are known compounds, their synthesis being described in, for example, U.S. Pat. Nos. 4,304,705; 5,081,197; and 5,091,489, the content of which are incorporated by reference herein. Suitable 2-alkenyl azlactones include, but are not limited to, 2-ethenyl-1,3-oxazolin-5-one, 2-ethenyl-4-methyl-1,3-oxazolin-5-one, 2-isopropenyl-1,3-oxazolin-5-one, 2-isopropenyl-4-methyl-1,3-oxazolin-5-one, 2-ethenyl-4,4-dimethyl-1,3-oxazolin-5-one, 2-isopropenyl-4, -dimethyl-1,3-oxazolin-5-one, 2-ethenyl-4-methyl-ethyl-1,3-oxazolin-5-one, 2-isopropenyl-4-methyl-4-butyl-1,3-oxazolin-5-one, 2-ethenyl-4,4-dibutyl-1,3-oxazolin-5-one, 2-isopropenyl-4-methyl-4-dodecyl-1,3-oxazolin-5-one, 2-isopropenyl-4,4-diphenyl-1,3-oxazolin-5-one, 2-isopropenyl-4,4-pentamethylene-1,3-oxazolin-5-one, 2-isopropenyl-4,4-tetramethylene-1,3-oxazolin-5-one, 2-ethenyl-4,4-diethyl-1,3-oxazolin-5-one, 2-ethenyl-4-methyl-4-nonyl-1,3-oxazolin-5-one, 2-isopropenyl-methyl-4-phenyl-1,3-oxazolin-5-one, 2-isopropenyl-4-methyl-4-benzyl-1,3-oxazolin-5-one, and 2-ethenyl-4,4-pentamethylene-1,3-oxazolin-5-one.

In an illustrative embodiment, the azlactone monomers can be represented by the following general formula:

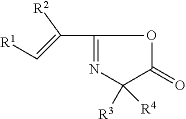

where R¹ and R² independently denote a hydrogen atom or a lower alkyl radical with one to six carbon atoms, and R³ and R+independently denote alkyl radicals with one to six carbon atoms or a cycloalkyl radical with five or six carbon atoms. Specific examples include 2-isopropenyl-4,4-dimethyl-2-oxazolin-5-one (IPDMO), 2-vinyl-4,4-dimethyl-2-oxazolin-5-one (VDMO), spiro-4′-(2′-isopropenyl-2′-oxazolin-5-one) cyclohexane (IPCO), cyclohexane-spiro-4′-(2′-vinyl-2′-oxazol-5′-one) (VCO), and 2-(-1-propenyl)-4,4-dimethyl-oxazol-5-one (PDMO) and the like. These compounds and their preparation are known in the art, see, e.g., U.S. Pat. No. 6,858,310, the contents of which are incorporated by reference herein.

In one illustrative embodiment, the ethylenically unsaturated containing monomer having ring-opening reactive functionalities is a non-boronic acid-containing ethylenically unsaturated containing monomer having ring-opening reactive functionalities.

The block copolymers further include monomeric units derived from an ethylenically unsaturated-containing hydrophilic monomer. The ethylenically unsaturated-containing hydrophilic monomers include, for example, ethylenically unsaturated-containing hydrophilic monomer comprises moieties that include for example, acrylamides such as N,N-dimethylacrylamide, N,N-dimethylmethacrylamide, and the like; acetamides such as N-vinyl-N-methyl acetamide, N-vinyl acetamide and the like; formamides such as N-vinyl-N-methyl formamide, N-vinyl formamide, and the like; cyclic lactams such as N-vinyl-2-pyrrolidone and the like; (meth)acrylated alcohols such as 2-hydroxyethyl methacrylate, 2-hydroxyethyl acrylate and the like; ethylenically unsaturated polymerizable alkoxylated polymers and the like and mixtures thereof. Methacrylated sulfobetaines and methacrylated phosphocholines are also contemplated herein.

Suitable ethylenically unsaturated polymerizable alkoxylated polymers include, by way of example, polymerizable polyethylene glycols having a number average molecular weight of up to, for example, about 2000 such as those with CTFA names PEG-200, PEG-400, PEG-600, PEG-1000, and mixtures thereof. Representative examples include, but are not limited to, (meth)acrylated poly(ethyleneglycol)s such as PEG-200 methacrylate, PEG-400 methacrylate, PEG-600 methacrylate, PEG-1000 methacrylate and the like and mixtures thereof.

In one illustrative embodiment, the ethylenically unsaturated-containing hydrophilic monomer is a non-boronic acid-containing ethylenically unsaturated-containing hydrophilic monomer.

The block copolymers disclosed herein can be prepared using techniques of controlled radical polymerization, e.g., by reversible addition-fragmentation chain transfer (RAFT) polymerization or atom-transfer radical polymerization (ATRP) employing a chain transfer agent that allows construction of block copolymers with a well-defined molecular weight distribution and narrow polydispersity. RAFT polymerization is particularly preferred because it is compatible with a wide variety of vinyl monomers.

In non-limiting illustrative embodiments, the RAFT agents suitable for use herein can be based upon thio carbonyl thio chemistry which is well known to those of ordinary skill in the art. The thio carbonyl thio fragment can be derived from a RAFT agent such as, for example, a xanthate-containing compound, trithiocarbonate-containing compound, dithiocarbamate-containing compound, a dithiobenzoate-containing compound or dithio ester-containing compound, wherein each compound contains a thio carbonyl thio group. One class of RAFT agents that can be used herein is of the general formula:

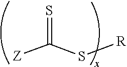

wherein x is 1 or 2, Z is a substituted oxygen (e.g., xanthates (—O—R)), a substituted nitrogen (e.g., dithiocarbamates (—NRR)), a substituted sulfur (e.g., trithiocarbonates (—S—R)), a dithiobenzoate, a substituted or unsubstituted C₁-C₂₀ alkyl or C₃-C₂₅ unsaturated, or partially or fully saturated ring (e.g., dithioesters (—R)) or carboxylic acid-containing group; and R is independently a straight or branched, substituted or unsubstituted C₁-C₃₀ alkyl cyano group, a straight or branched, substituted or unsubstituted C₁-C₃₀ alkyl group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a C₁-C₂₀ ester group; an ether or polyether-containing group; an alkyl- or arylamide group; an alkyl- or arylamine group; a substituted or unsubstituted C₅-C₃₀ heteroaryl group; a substituted or unsubstituted C₃-C₃₀ heterocyclic ring; a substituted or unsubstituted C₄-C₃₀ heterocycloalkyl group; a substituted or unsubstituted C₆-C₃₀ heteroarylalkyl group; and combinations thereof.

Representative examples of alkyl groups for use herein include, by way of example, a straight or branched alkyl chain radical containing carbon and hydrogen atoms of from 1 to about 30 carbon atoms or from 1 to about 12 carbon atoms with or without unsaturation, to the rest of the molecule, e.g., methyl, ethyl, n-propyl, 1-methylethyl (isopropyl), n-butyl, n-pentyl, methylene, ethylene, etc., and the like.

Representative examples of cycloalkyl groups for use herein include, by way of example, a substituted or unsubstituted non-aromatic mono or multicyclic ring system of about 3 to about 30 carbon atoms or from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 30 carbon atoms or from 3 to about 6 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 30 carbon atoms or from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 30 carbon atoms or from 5 to about 8 carbon atoms such as, for example, phenyl, naphthyl, tetrahydronapthyl, indenyl, biphenyl and the like, optionally containing one or more heteroatoms, e.g., O and N, and the like.

Representative examples of arylalkyl groups for use herein include, by way of example, a substituted or unsubstituted aryl group as defined herein directly bonded to an alkyl group as defined herein, e.g., —CH₂C₆H₅, —C₂H₅C₆H₅ and the like, wherein the aryl group can optionally contain one or more heteroatoms, e.g., O and N, and the like.

Representative examples of ester groups for use herein include, by way of example, a carboxylic acid ester having one to 20 carbon atoms and the like.

Representative examples of ether or polyether containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are as defined herein. Exemplary ether or polyether-containing groups include, by way of example, alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures or copolymers thereof, an ether or polyether group of the general formula —(R²OR³), wherein R² is a bond, a substituted or unsubstituted alkyl, cycloalkyl or aryl group as defined herein and R³ is a substituted or unsubstituted alkyl, cycloalkyl or aryl group as defined herein and tis at least 1, e.g., —CH₂CH₂OC₆H₅ and CH₂—CH₂—CH₂—O—CH₂—(CF₂)_{z}—H where z is 1 to 6, —CH₂CH₂OC₂H₅, and the like.

Representative examples of alkyl or arylamide groups for use herein include, by way of example, an amide of the general formula —R⁴C(O)NR⁵R⁶ wherein R⁴, R⁵ and R⁶ are independently C₁—C₃₀ hydrocarbons, e.g., R⁴ can be alkylene groups, arylene groups, cycloalkylene groups and R⁵ and R⁶ can be alkyl groups, aryl groups, and cycloalkyl groups as defined herein and the like.

Representative examples of alky or arylamine groups for use herein include, by way of example, an amine of the general formula —R⁷NR⁸R⁹ wherein R⁷ is a C₂-C₃₀ alkylene, arylene, or cycloalkylene and R⁸ and R⁹ are independently C₁-C₃₀ hydrocarbons such as, for example, alkyl groups, aryl groups, or cycloalkyl groups as defined herein.

Representative examples of heterocyclic ring groups for use herein include, by way of example, a substituted or unsubstituted stable 3 to about 30 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic).

Representative examples of heteroaryl groups for use herein include, by way of example, a substituted or unsubstituted heterocyclic ring radical as defined herein. The heteroaryl ring radical may be attached to the main structure at any heteroatom or carbon atom that results in the creation of a stable structure.

Representative examples of heteroarylalkyl groups for use herein include, by way of example, a substituted or unsubstituted heteroaryl ring radical as defined herein directly bonded to an alkyl group as defined herein. The heteroarylalkyl radical may be attached to the main structure at any carbon atom from the alkyl group that results in the creation of a stable structure.

Representative examples of heterocyclic groups for use herein include, by way of example, a substituted or unsubstituted heterocylic ring radical as defined herein. The heterocyclic ring radical may be attached to the main structure at any heteroatom or carbon atom that results in the creation of a stable structure.

Representative examples of heterocycloalkyl groups for use herein include, by way of example, a substituted or unsubstituted heterocylic ring radical as defined herein directly bonded to an alkyl group as defined herein. The heterocycloalkyl radical may be attached to the main structure at any carbon atom in the alkyl group that results in the creation of a stable structure.

The substituents in the ‘substituted oxygen’, ‘substituted nitrogen’, ‘substituted sulfur’, ‘substituted alkyl’, ‘substituted alkylene’, ‘substituted cycloalkyl’, ‘substituted cycloalkylalkyl’, ‘substituted cycloalkenyl’, ‘substituted arylalkyl’, ‘substituted aryl’, ‘substituted heterocyclic ring’, ‘substituted heteroaryl ring,’ ‘substituted heteroarylalkyl’, ‘substituted heterocycloalkyl ring’, ‘substituted cyclic ring’ may be the same or different and include one or more substituents such as hydrogen, hydroxy, halogen, carboxyl, cyano, nitro, oxo (═O), thio(═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocycloalkyl ring, substituted or unsubstituted heteroarylalkyl, substituted or unsubstituted heterocyclic ring, and the like.

Representative examples of RAFT agents for use herein include, but are not limited to, -cyano-4-(dodecyl-sulfanylthiocarbonyl)sulfanylpentanoic acid, S-cyanomethyl-5-dodecyltrithiocarbonate, S-(2-cyano-2-propyl)-S-dodecyltrithiocarbonate, 3-benzylsulfanylthiocarbonylsulfanyl-propionic acid, cumyl dithiobenzoate, 2-cyanoprop-2-yl dithiobenzoate (i.e., cyanoisopropyl dithiobenzoate), 4-thiobenzoylsulfanyl-4-cyanopentanoic acid (TCA), S,S′-bis(α,α′-dimethyl-alpha″-acetic acid)-trithiocarbonate (BATC), benzyl dodecyl trithiocarbonate, ethyl-2-dodecyl trithiocarbony) proprionate, S-sec propionic acid O-ethyl xanthate, α-ethyl xanthylphenylacetic acid, ethyl α-(o-ethyl xanthyl) proprionate, ethyl α-(ethyl xanthyl) phenyl acetate, ethyl 2-(dodecyl trithiocarbonyl) phenyl acetate, ethyl 2-(dodecyl trithiocarbonyl) propionate, 2-(dodecylthiocarbonylthiol)propanoic acid, and the like and mixtures thereof.

There is no particular limitation on the organic chemistry used to form the RAFT agent and is within the purview of one skilled in the art. Also, the working examples below provide guidance. For example, the RAFT agents can be prepared as exemplified in Schemes I-VII below.

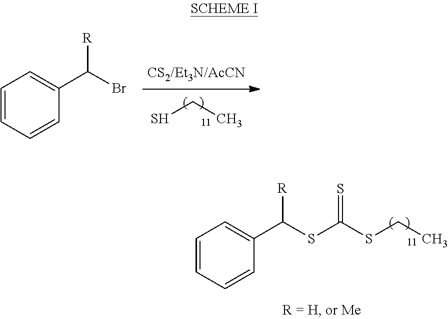

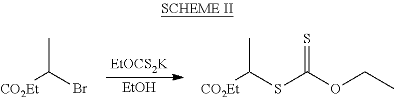

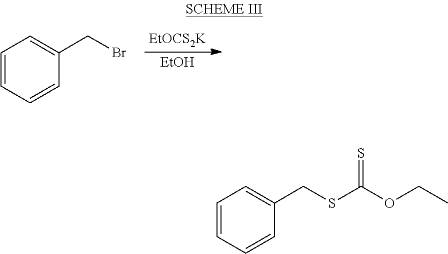

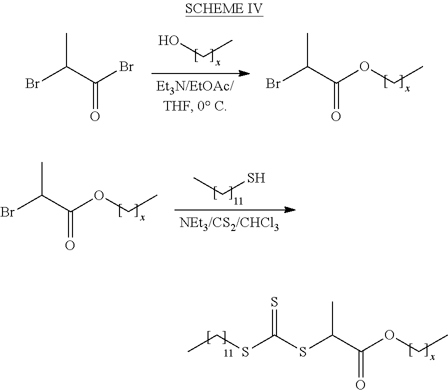

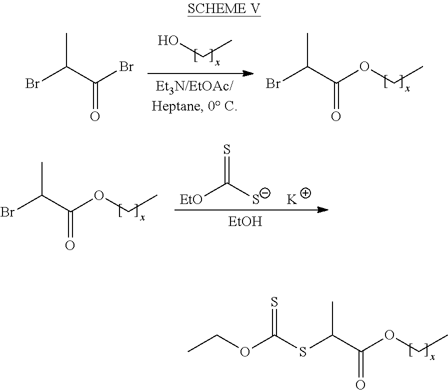

The block copolymers disclosed herein can be obtained in a first step (a) by (1) mixing either the ethylenically unsaturated containing monomer having ring-opening reactive functionalities or the ethylenically unsaturated-containing hydrophilic monomer with a RAFT agent; (2) adding a polymerization initiator; (3) and subjecting the monomer/RAFT agent/initiator mixture to a source of heat. Suitable initiators include, for example, free-radical-generating polymerization initiators of the type illustrated by acetyl peroxide, lauroyl peroxide, decanoyl peroxide, coprylyl peroxide, benzoyl peroxide, tertiary butyl peroxypivalate, sodium percarbonate, tertiary butyl peroctoate, and azobisisobutyronitrile (AIBN).

The reaction can be carried out at a temperature of between about 15° C. to about 120° C. for a time period of about 30 minutes to about 48 hours. If desired, the reaction can be carried out in the presence of a suitable solvent. Suitable solvents are in principle all solvents which dissolve the monomer used, for example, 1,4-dioxane, hexanol, dimethylformamide; acetone, cyclohexanone, toluene, and the like and mixtures thereof.

In an illustrative embodiment, the ethylenically unsaturated containing monomer having ring-opening reactive functionalities or the ethylenically unsaturated-containing hydrophilic monomer is employed in an amount ranging from about 10 to about 50 weight percent, based on the total weight of the mixture. In an illustrative embodiment, the RAFT agent is employed in an amount ranging from about 0.5 to about 3 weight percent, based on the total weight of the mixture. The level of initiator employed will vary within the range of 0.01 to 2 weight percent of the mixture of monomers. If desired, the mixture of the above-mentioned monomers is warmed with addition of a free-radical former.

Next, in a step (b) the resulting product of step (a) is then mixed with the other one of the ethylenically unsaturated containing monomer having ring-opening reactive functionalities or the ethylenically unsaturated-containing hydrophilic monomer and an initiator and subjected to a source of heat as described above until the desired block copolymer is formed. In an illustrative embodiment, the other one of the ethylenically unsaturated containing monomer having ring-opening reactive functionalities or the ethylenically unsaturated-containing hydrophilic monomer is employed in an amount ranging from about 10 to about 50 weight percent, based on the total weight of the mixture. In an illustrative embodiment, the resulting product of step (a) is employed in an amount ranging from about 1 to about 20 weight percent based on the total weight of the mixture.

A non-limiting schematic representation of a synthetic method for making the block copolymer with a RAFT agent is set forth below in Scheme VI.

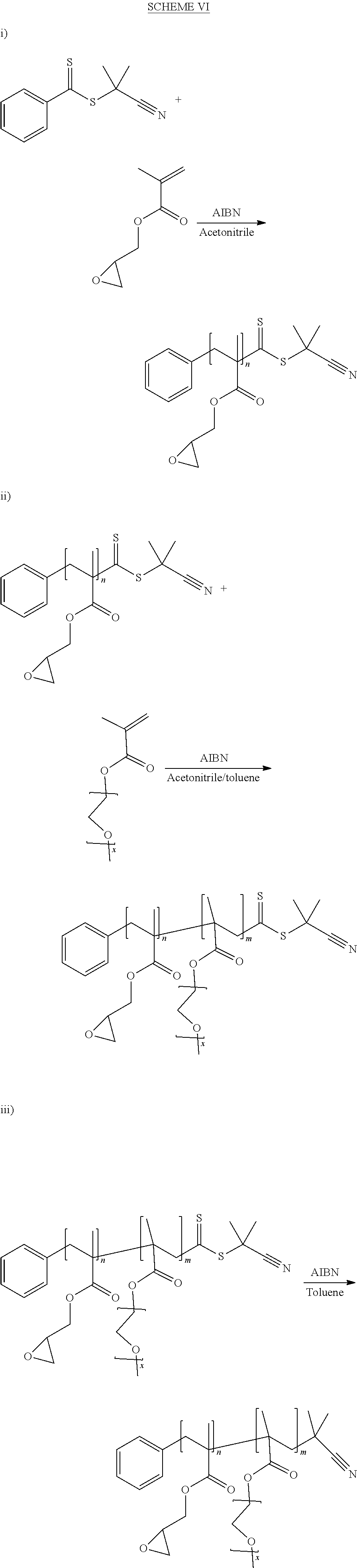

where m is about 50 to about 300, n is 1 to about 30 and x is about 100 to about 1000.

In the case where the block copolymer disclosed herein is obtained from ATRP polymerization, the ethylenically unsaturated groups may be introduced by appropriate selection of a suitable ATRP initiator or by displacement reactions of the terminal halogen atom. Suitable ATRP groups for use herein include any standard monofunctional or difunctional ATRP group as is well known to those of ordinary skill in the art. A comprehensive review on the use of ATRP initiators or displacement of the terminal halogen using electrophilic, nucleophilic, and radical reactions to produce telechelic polymers is disclosed in, for example, Matyjaszewski, K.; Xia, J. Chem. Rev., 101, 2921-2990 (2001).

In one embodiment, a useful ATRP group includes an ethylenically unsaturated ATRP initiator such as, for example, vinyl functionalized ATRP initiators, e.g., prop-2-enyl-2′-bromoisobutyrate, vinyl chloroacetate, allyl chloroacetate, allyl bromide and the like. These initiators are used to polymerize either hydrophilic monomers or hydrophobic monomers.

In another embodiment, a useful ATRP group includes a non-ethylenically unsaturated ATRP initiator that can be converted to an ethylenically unsaturated initiator by a subsequent step. Examples of such initiators include α-bromo-isobutyric acid, hydroxyethyl 2-bromopropionate, glycidol 2-bromopropionate, tert-butyl 2-bromopropionate, and 4-bromobenzyl bromide, and the like.

In an illustrative embodiment, the block copolymer is obtained from ATRP polymerization in a first step (a) by (1) mixing either the ethylenically unsaturated containing monomer having ring-opening reactive functionalities or the ethylenically unsaturated-containing hydrophilic monomer with, for example, an ATRP initiator and suitable ATRP catalyst such as a copper(I) bromide and subjecting the monomer/ATRP agent/initiator mixture to a source of heat. The reaction can be carried out at a temperature of between about 15° C. to about 120° C. for a time period of about 30 minutes to about 48 hours. If desired, the reaction can be carried out in the presence of a suitable solvent. Suitable solvents are in principle all solvents which dissolve the monomers used, for example, 1,4-dioxane, hexanol, dimethylformamide; acetone, cyclohexanone, toluene, and the like and mixtures thereof.

In an illustrative embodiment, the ethylenically unsaturated containing monomer having ring-opening reactive functionalities or the ethylenically unsaturated-containing hydrophilic monomer is employed in an amount ranging from about 90 wt. % to about 99 wt. %, based on the total weight of the mixture. In an illustrative embodiment, the ATRP initiator is employed in an amount ranging from about 0.5 wt. % to about 10 wt. %, based on the total weight of the mixture. The level of catalyst employed will vary within the range of 0.01 to 2 weight percent of the mixture of monomers.

Next, in a step (b) the resulting product of step (a) is then mixed with the other one of the ethylenically unsaturated containing monomer having ring-opening reactive functionalities or the ethylenically unsaturated-containing hydrophilic monomer and an initiator and subjected to a source of heat as described above until the desired block copolymer is formed. In an illustrative embodiment, the other one of the ethylenically unsaturated containing monomer having ring-opening reactive functionalities or the ethylenically unsaturated-containing hydrophilic monomer is employed in an amount ranging from about 90 wt. % to about 99 wt. %, based on the total weight of the mixture. In an illustrative embodiment, the resulting product of step (a) is employed in an amount ranging from about 0.1 wt. % to about 10 wt. %, based on the total weight of the mixture.

The reaction can be carried out at a temperature of between about 50° C. to about 150° C. for about 1 to about 48 hours. The reaction can be carried out in the presence of a suitable solvent as discussed above.

A non-limiting schematic representation of a synthetic method for making the block copolymer with an ATRP agent is set forth below in Scheme VII.

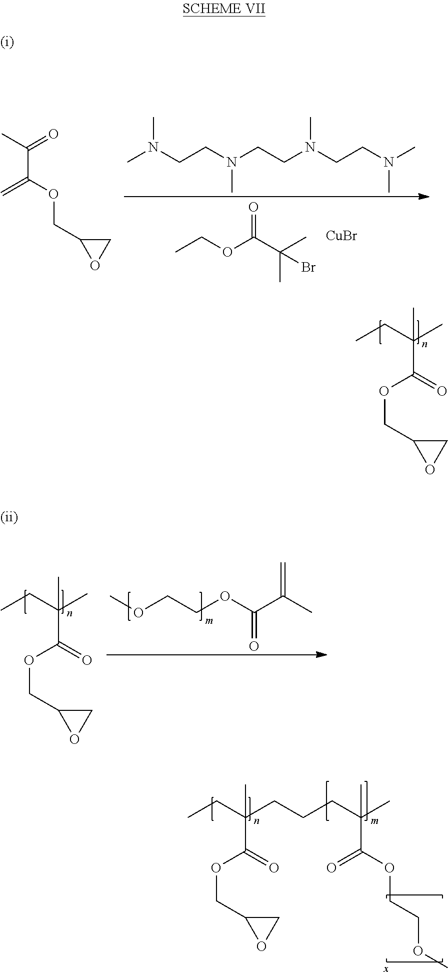

where m is about 50 to about 300, n is 1 to about 30 and x is about 100 to about 1000.

As one skilled in the art will readily appreciate, the block copolymer will contain a balance of monomeric units derived from the ethylenically unsaturated containing monomer having ring-opening reactive functionalities and monomeric units derived from the ethylenically unsaturated-containing hydrophilic monomer. In non-limiting illustrative embodiments, the number of monomeric units of the block copolymer derived from ethylenically unsaturated containing monomers having ring-opening reactive functionalities can be from about 10 to about 60 units. In another non-limiting illustrative embodiment, the number of monomeric units of the block copolymer derived from an ethylenically unsaturated containing monomers having ring-opening reactive functionalities can be from about 15 to about 45 units. In another non-limiting illustrative embodiment, the number of monomeric units of the block copolymer derived from an ethylenically unsaturated containing monomers having ring-opening reactive functionalities can be from about 15 to about 30 units.

In non-limiting illustrative embodiments, the number of monomeric units of the block copolymer derived from an ethylenically unsaturated-containing hydrophilic monomer can be from about 70 to about 250 units. In another non-limiting illustrative embodiment, the number of monomeric units of the block copolymer derived from an ethylenically unsaturated-containing hydrophilic monomer can be from about 100 to about 200 units. In another non-limiting illustrative embodiment, the number of monomeric units of the block copolymer derived from an ethylenically unsaturated-containing hydrophilic monomer can be from about 125 to about 175 units.

Any combination of the forgoing ranges of numbers of monomeric units of the block copolymer derived from ethylenically unsaturated containing monomers having ring-opening reactive functionalities and the number of monomeric units of the block copolymer derived from an ethylenically unsaturated-containing hydrophilic monomer are contemplated herein.

In one or more additional non-limiting illustrative embodiments, as may be combined with one or more of the preceding paragraphs, the biomedical device having one or more biomedical device surface reactive functional groups and/or one or more biomedical device surface protected reactive functional groups are then exposed to the block copolymer disclosed herein to form a surface coating on the biomedical device.

In a non-limiting illustrative embodiment, the step of forming the surface coating can comprise diffusing the block copolymer in the biomedical device. For example, the step of diffusing the block copolymer in the biomedical device can include at least (a) soaking the biomedical device in a swelling solution comprising one or more solvents and the block copolymer; and (b) removing the biomedical device from the swelling solution to provide the biomedical device comprising the block copolymer diffused in the biomedical device. In other words, diffusion of the block polymer in the biomedical device is accomplished through the swelling of the biomedical device to allow the block copolymer to diffuse in and become entrapped in the biomedical device once swelling is reversed.

In an illustrative embodiment, step (a) above includes soaking the biomedical device in one or more solvent solutions and the block copolymer for a time period sufficient to swell the biomedical device. In general, the one or more solvent solutions can include, for example, a solvent capable of swelling the biomedical device and at same time can solubilize the block copolymer. In one embodiment, the one or more solvent solutions include, for example, a low molecular weight alcohol solvent, an aliphatic hydrocarbon solvent, a cycloaliphatic hydrocarbon solvent, a ketone solvent, a nitrile solvent, an amido group-containing solvent and mixtures thereof. Suitable low molecular weight alcohols include, for example, low molecular weight alcohols having about 1 to about 13 carbon atoms and/or a molecular weight of no greater than about 200. A suitable low molecular alcohol can be selected from a variety of low-molecular-weight monohydric alcohols, each comprising about 1 to about 13 carbon atoms. Suitable monohydric alcohols include, for example, methanol, ethanol, propanol, isopropyl alcohol, butanol, isobutyl alcohol, tert-butyl alcohol, hexanol, 2-ethylhexanol, dodecanol, and the like. Suitable aliphatic or cycloaliphatic hydrocarbon solvents include, for example, pentane, hexane, heptane, cyclohexane and the like.

Suitable ketone solvents include, for example, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and combinations thereof. In one embodiment, a ketone solvent is acetone.

Suitable nitrile solvents include, for example, saturated or unsaturated aliphatic, alicyclic, or aromatic compounds containing a nitrile group. Included within the nitriles are compounds containing heteroatom such as those selected from Groups 13, 14, 15, 16 and 17 of the Periodic Table of Elements. Representative examples of nitriles for use herein include acetonitrile; propionitrile; isopropionitrile; butyronitrile; isobutyronitrile; valeronitrile; isovaleronitrile; trimethylacetonitrile; hexanenitrile; heptanenitrile; heptyl cyanide; octyl cyanide; undecanenitrile; malononitrile; succinonitrile; glutaronitrile; adiponitrile; sebaconitrile; allyl cyanide; acrylonitrile; crotononitrile; methacrylonitrile; fumaronitrile; tetracyanoethylene; cyclopentanecarbonitrile; cyclohexanecarbonitrile; dichloroacetonitrile; fluoroacetonitrile; trichloroacetonitrile; benzonitrile; benzyl cyanide; 2-methylbenzyl cyanide; 2-chlorobenzonitrile; 3-chlorobenzonitrile; 4-chlorobenzonitrile; o-tolunitrile; m-tolunitrile; p-tolunitrile and the like and mixtures thereof. In one embodiment, a nitrile solvent is acetonitrile.

Suitable amide group-containing solvents include, for example, dimethyl formamide, N-methyl formanilide, N-formyl piperidine, N-formyl morpholine, dimethyl acetamide, N-methyl pyrrolidone, N,N-dimethyl benzamide and mixtures thereof. In one embodiment, an amido group-containing solvent is N-methyl pyrrolidone.

In one embodiment, the one or more solvent solutions can further include water in combination with any of the foregoing solvents. For example, the one or more solvent solutions can be a blend containing from about 25 wt. % to about 75 wt. % one or more solvent solutions and from about 75 wt. % to about 25 wt. % water. In another embodiment, a blend can contain from about 40 wt. % to about 60 wt. % one or more solvent solutions and from about 60 wt. % to about 40 wt. % water.

The biomedical device is soaked in the one or more solvent solutions and block copolymer for a time period sufficient to swell the biomedical device. In an embodiment, the biomedical device is soaked in the one or more solvent solutions and block copolymer for a time period ranging from about 5 minutes to about 120 minutes. In one embodiment, the biomedical device is soaked in the one or more solvent solutions and block copolymer for a time period ranging from about 5 minutes to about 60 minutes. In one embodiment, the biomedical device is soaked in the one or more solvent solutions and block copolymer for a time period ranging from about 10 minutes to about 35 minutes.

Once the biomedical device has been swelled the block copolymer is diffused into the swelled biomedical device. Next, the biomedical device is removed from the solvent solution to provide the biomedical device having the block copolymer diffused therein and forming a surface coating on the biomedical device. In step (b), the swelled biomedical device can be soaked in one or more solvent solutions comprising the block copolymer to de-swell the biomedical device and entrap the block copolymer in the biomedical device. Suitable solvent solutions include, for example, water and any of the foregoing low molecular weight alcohol solvent, aliphatic hydrocarbon solvent, cycloaliphatic hydrocarbon solvent, ketone solvent, nitrile solvent, ether solvent, and amido group-containing solvents discussed hereinabove.

After the biomedical device has been de-swelled, the biomedical device is removed and optionally soaked in one more series of water solutions to further de-swell the biomedical device. In general, the biomedical device can be soaked in the one or more water solutions for a time period ranging from about 5 minutes to about 20 minutes.

Next, the de-swelled biomedical device is sterilized. In one embodiment, the de-swelled biomedical device is sterilized by submerging the de-swelled biomedical device in a borate buffered saline and then subjecting it to autoclave conditions for at least about 5 minutes, or at least about 20 minutes or at least 24 hours or up to about 72 hours. The sterilized biomedical device is then rinsed with water and positioned in its packaging with borate buffered saline. The package is sealed and again the biomedical device is subjected to autoclave conditions.

Alternatively, the de-swelled biomedical device can be placed in a container that includes a receptacle portion to hold the de-swelled ophthalmic device and a sterile packaging solution. Examples of the container are conventional biomedical device blister packages. This receptacle, containing the de-swelled biomedical device immersed in the solution, is hermetically sealed, for example, by sealing lidstock on the package over the receptacle. For example, the lidstock is sealed around a perimeter of the receptacle. The solution and the de-swelled biomedical device are sterilized while sealed in the package receptacle. Examples of sterilization techniques include subjecting the solution and the de-swelled ophthalmic device to thermal energy, microwave radiation, gamma radiation or ultraviolet radiation. A specific example involves heating the solution and the de-swelled ophthalmic device, while sealed in the package container, to a temperature of at least 100° C., or at least 120° C., such as by autoclaving.

In another non-limiting illustrative embodiment, the step of forming the surface coating can comprise exposing the biomedical device having one or more biomedical device surface reactive functional groups and/or one or more biomedical device surface protected reactive functional groups to the block copolymer comprising (a) monomeric units derived from an ethylenically unsaturated containing monomer having ring-opening reactive functionalities or nucleofugic functionalities that are complementary to the one or more biomedical device surface reactive functional groups and/or one or more biomedical device surface protected reactive functional groups, and (b) monomeric units derived from an ethylenically unsaturated-containing hydrophilic monomer to adsorb, entangle or covalently attach the ring-opening reactive functionalities of the monomeric units derived from an ethylenically unsaturated containing monomer having ring-opening reactive functionalities of the block copolymer to the one or more biomedical device surface reactive functional groups and/or the one or more biomedical device surface protected reactive functional groups of the biomedical device. Entanglement of the block copolymer to the biomedical device is understood to mean structures formed by the cross-linking points between intermolecular or intramolecular polymer chains, making the polymer chains unable to move normally and thus trapping them into the bulk matrix. In addition, absorption of the block copolymer to the biomedical device lens involves intermolecular forces brought about by electrostatic interaction, hydrogen bonding, and van der Waals forces.

In non-limiting illustrative embodiments, the biomedical device can be released from a mold assembly and then contacted with an aqueous packaging solution containing the block copolymer disclosed herein. For example, the biomedical device can be transferred to an individual lens package containing a buffered saline solution containing at least the block copolymers disclosed herein and subjected to sterilization. In non-limiting illustrative embodiments, the block copolymers disclosed herein are present in the aqueous packaging solution in an amount ranging from about 0.01 to about 3 wt. %, based on the total weigh of the aqueous packaging solution. In other non-limiting illustrative embodiments, the block copolymers disclosed herein are present in the aqueous packaging solution in an amount ranging from about 0.01 to about 1.5 wt. %, based on the total weigh of the aqueous packaging solution. In yet other non-limiting illustrative embodiments, the block copolymers disclosed herein are present in the aqueous packaging solution in an amount ranging from about 0.01 to about 1 wt. %, based on the total weigh of the aqueous packaging solution. In yet other non-limiting illustrative embodiments, the block copolymers disclosed herein are present in the aqueous packaging solution in an amount ranging from about 0.05 to about 0.5 wt. %, based on the total weigh of the aqueous packaging solution.

Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, steam sterilizing or autoclaving of the sealed container at temperatures of about 120° C. or higher.

The packaging solutions of the illustrative embodiments are physiologically compatible. Specifically, the solution must be “ophthalmically safe” for use with a lens such as a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The packaging solution should also be sterile in that the absence of microbial contaminants in the product prior to release must be statistically demonstrated to the degree necessary for such products. The liquid media useful in the present invention are selected to have no substantial detrimental effect on the lens being treated or cared for and to allow or even facilitate the present lens treatment or treatments. The liquid media are preferably aqueous-based. A particularly useful aqueous liquid medium is that derived from saline, for example, a conventional saline solution or a conventional buffered saline solution.

The pH of the present solutions is maintained within the range of about 6 to about 9, and preferably about 6.5 to about 7.8. As mentioned above, additional buffer may optionally be added, such as boric acid, sodium borate, potassium citrate, sodium citrate, citric acid, sodium bicarbonate, various mixed phosphate buffers (including combinations of Na₂ HPO₄, NaH₂PO₄ and KH₂ PO₄), hydrates thereof and the like and mixtures thereof. Generally, buffers will be used in amounts ranging from about 0.05 to about 2.5 percent by weight, and preferably from about 0.1 to about 1.5 percent by weight of the solution. However, according to certain embodiments, tris(hydroxymethyl)aminomethane, or salts thereof, function as the sole buffer.

In one embodiment, the aqueous packaging solution can further comprise one or more buffer agents. Suitable one or more buffer agents include, for example, phosphate buffer agents, borate buffer agents, citrate buffer agents, and the like. A suitable phosphate buffer agent can be any known phosphate buffer agents. In one embodiment, the phosphate buffer agent comprises one or more of sodium hydrogen phosphate monobasic, sodium hydrogen phosphate dibasic, potassium hydrogen phosphate monobasic and potassium hydrogen phosphate dibasic and any suitable hydrate thereof, e.g., monohydrate and heptahydrate. A suitable borate buffer agent can be any known borate buffer agents. In one embodiment, the borate buffer agent comprises one or more of boric acid and sodium borate. A suitable citrate buffer agent can be any known citrate buffer agents. In one embodiment, the citrate buffer agent comprises one or more of citric acid and sodium citrate.

In one embodiment, the one or more buffer agents are present in the aqueous packaging solution in an amount ranging from about 0.001 to about 2 wt. %, based on the total weight of the packaging solution. In one embodiment, the phosphate buffer agent is present in the packaging solution in an amount ranging from about 0.001 to about 1 wt. %, based on the total weight of the packaging solution.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Examples of suitable tonicity adjusting agents include, but are not limited to, sodium and potassium chloride, dextrose, glycerin, calcium and magnesium chloride and the like and mixtures thereof. These agents are typically used individually in amounts ranging from about 0.01 to about 2.5% w/v and preferably from about 0.2 to about 1.5% w/v. Preferably, the tonicity agent will be employed in an amount to provide a final osmotic value of at least about 200 mOsm/kg, or from about 200 to about 400 mOsm/kg, or from about 250 to about 350 mOsm/kg, or from about 280 to about 320 mOsm/kg.

If desired, one or more additional components can be included in the packaging solution. Such an additional component or components are chosen to impart or provide at least one beneficial or desired property to the packaging solution. Such additional components may be selected from components which are conventionally used in one or more ophthalmic device care compositions. Examples of such additional components include cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

Useful sequestering agents include, but are not limited to, disodium ethylene diamine tetraacetate, alkali metal hexametaphosphate, citric acid, sodium citrate and the like and mixtures thereof.

Useful viscosity builders include, but are not limited to, hydroxyethyl cellulose, hydroxymethyl cellulose, polyvinyl pyrrolidone, polyvinyl alcohol and the like and mixtures thereof.

Useful antioxidants include, but are not limited to, sodium metabisulfite, sodium thiosulfate, N-acetylcysteine, butylated hydroxyanisole, butylated hydroxytoluene and the like and mixtures thereof.

The method of packaging and storing a biomedical device, e.g., an ophthalmic device such as a contact lens includes at least packaging an ophthalmic device immersed in the aqueous packaging solution containing the block copolymer disclosed herein and sterilizing the packaged solution. The method may include immersing the ophthalmic device in the aqueous packaging solution prior to delivery to the customer/wearer, directly following manufacture of the contact lens. Alternately, the packaging and storing in the solution of the present invention may occur at an intermediate point before delivery to the ultimate customer (wearer) but following manufacture and transportation of the lens in a dry state, wherein the dry lens is hydrated by immersing the lens in the packaging solution. Consequently, a package for delivery to a customer may include a sealed container containing one or more unused contact lenses immersed in an aqueous packaging solution.

In one embodiment, an illustration of the method disclosed herein is generally depicted below in Scheme I below.

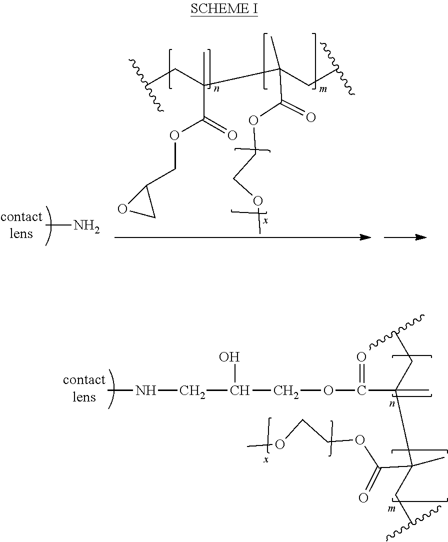

wherein n is from about 10 to about 60, m is from about 70 to about 250 and x is from about 100 to about 1000.

After polymerization is completed, any non-covalently bonded monomers, oligomers or polymers formed can be removed, for example, by treatment with a suitable solvent. The resulting surface modified biomedical device can then be used “as is”. In other words, no additional surface treatment steps will have to be carried out to modify the resulting surface modified biomedical device. As used herein, the phrase “without any additional surface treatment steps” shall be understood to mean that the exterior surface of the surface modified biomedical device of the present invention is not further treated to modify the surface thereof by, for example, oxidation treatments, plasma treatments, grafting treatments, coating treatments and the like. However, it shall be understood that coatings such as color or other cosmetic enhancement may be applied to devices disclosed herein.

The following examples are provided to enable one skilled in the art to practice the invention and are merely illustrative of the invention. The examples should not be read as limiting the scope of the invention as defined in the claims.

Various block copolymers were formed and then coated onto a contact lens as discussed below and characterized by standard testing procedures such as:

Contact Angle: Captive bubble contact angle data was collected on a First Ten Angstroms FTA-1000 prop Shape Instrument. All samples were rinsed in HPLC grade water prior to analysis in order to remove components of the packaging solution from the sample surface. Prior to data collection the surface tension of the water used for all experiments was measured using the pendant drop method. In order for the water to qualify as appropriate for use, a surface tension value of 70-72 dynes/cm was expected. All lens samples were placed onto a curved sample holder and submerged into a quartz cell filled with HPLC grade water. Advancing and receding captive bubble contact angles were collected for each sample. The advancing contact angle is defined as the angle measured in water as the air bubble is retracting from the lens surface (water is advancing across the surface). All captive bubble data was collected using a high speed digital camera focused onto the sample/air bubble interface. The contact angle was calculated at the digital frame just prior to contact line movement across the sample/air bubble interface. The receding contact angle is defined as the angle measured in water as the air bubble is expanding across the sample surface (water is receding from the surface).

Sessile drop contact angle: Data was collected with a Kruss DSA instrument. Lenses are blotted dry with a kimwipe to get rid of excess water on the lens surface, and the lens is mounted on an orb-shaped mount. The cuvette is empty during testing, and the syringe is lowered from above and dispenses the water droplet onto the lens. Measurement is taken in the software and recorded.

Coefficient of Friction—The instrument used is a TA Discovery Hybrid controlled stress rheometer equipped with a custom ring tool of ˜50 nm surface roughness. A custom lens holder was designed to be used in an aqueous environment to eliminate effects of moisture loss from the surface.

### Method

The tool is lowered onto the lens surface until a target normal applied force F_{N} 0.05 N is reached. The programmed experiment is designed to measure the static, low speed kinetic and high speed kinetic frictional forces in 3 succinct steps.

Step 1: Static Coefficient of Friction - the rub tool torque is ramped at 2 μN·m/s from 0 to 500 μN·m while monitoring the rub tool velocity (v). When the torque applied to the rub tool exceeds the static friction force of the lens surface the tool begins to spin freely. The torque at the point the disk begins to spin, the radius of the disk, and the applied normal force are then utilized to calculate the static coefficient of friction.

Step 2: Low Speed Kinetic Coefficient of Friction—the rub tool is rotated at a constant ‘low’ speed and the tangential force recorded and averaged for 90 s to calculate the low speed kinetic CoF.

Step 3: High Speed Kinetic Coefficient of Friction—the rub tool rotation is increased for 30 s and again the tangential force is averaged and used to calculate the high speed kinetic CoF.

Sudan Black Staining Test

Step 1: Gently blot coated lenses to remove excess water.

Step 2: Place lenses in 0.2 wt. % solution of Sudan Black B in Mineral Oil and agitate for 10 minutes.

Step 3: Remove lenses from staining solution and vigorously rinse with water to remove excess stain and oil.

Step 4: Observe lens staining; areas which have stain adhered indicate a hydrophobic surface or incomplete coating.

### EXAMPLE 1A

Preparation of glycidyl methacrylate-poly (ethylene glycol) methacrylate block copolymer using ATRP block synthesis having a PEG portion of 210.

Glycidyl methacrylate (14.21 g, 0.100 mmol) was polymerized via ATRP using α-bromo-isobutyric acid initiator (0.39g, 2.00 mmol), 1,1,4,7,10, 10-hexamethyltriethylenetetramine ligand (0.023 g, 0.10 mmol), and copper(I) bromide catalyst (0.0143 g, 0.1 mmol). The glycidyl methacrylate, initiator and ligand were dissolved in 30 ml acetonitrile and purged with nitrogen for 1 hr. Catalyst was added and solution was purged with N₂ for 10 minutes. The solution was placed in a 45° C. oil bath and stirred for 2.5 hours. The solution was precipitated into methanol, filtered and dried under vacuum. Next, mPEG-methacrylate (500) (15 g, 30 mmol) was added to polyglycidyl methacrylate (0.161 g, 0.10 mmol) which had previously been dissolved in a small amount of acetone along with 30 ml of toluene. The solution was purged for 1 hr. under nitrogen. The reaction vessel was sealed and placed in a 60° C. oil bath for 8 hours. The reaction solution was concentrated and extracted with hexane 3 times. The residue was dissolved in acetone and passed through an aluminum oxide column. The polymer solution was precipitated in ethyl ether and dried in a vacuum oven at 30° C. overnight to remove the ether.

This reaction is generally shown below:

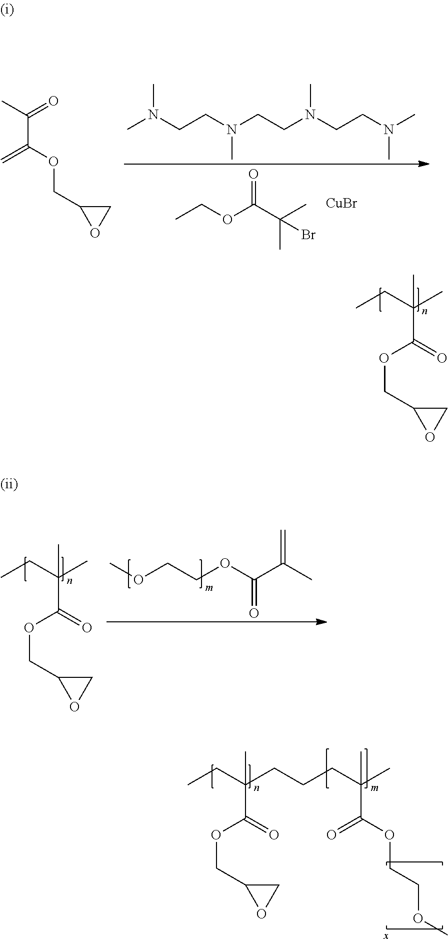

where m is 210, n is 15 and x is 500.

### EXAMPLE 1B

Preparation of glycidyl methacrylate-poly (ethylene glycol) methacrylate block copolymer using ATRP block synthesis having a PEG portion of 106.

Glycidyl methacrylate (14.21 g, 0.100 mmol) was polymerized via ATRP using α-bromo-isobutyric acid initiator (0.39 g, 2.00 mmol), 1,1,4,7,10,10-hexamethyltriethylenetetramine ligand (0.023 g, 0.10 mmol), and copper(I) bromide catalyst (0.0143 g, 0.1 mmol). The glycidyl methacrylate, initiator and ligand were dissolved in 30 ml acetonitrile and purged with nitrogen for 1 hr. Catalyst was added and solution was purged with N₂ for 10 minutes. The solution was placed in a 45° C. oil bath and stirred for 2.5 hours. The solution was precipitated into methanol, filtered and dried under vacuum. Next, mPEG-methacrylate (500) (7.50 g, 15 mmol) was added to polyglycidyl methacrylate (0.161 g, 0.10 mmol) which had previously been dissolved in a small amount of acetone along with 30 ml of toluene. The solution was purged for 1 hr. under nitrogen.

The reaction vessel was sealed and placed in a 60° C. oil bath for 8 hours. The reaction solution was concentrated and extracted with hexane 3 times. The residue was dissolved in acetone and passed through an aluminum oxide column. The polymer solution was precipitated in ethyl ether and dried in a vacuum oven at 30° C. overnight to remove the ether.

### EXAMPLE 2

Preparation of glycidyl methacrylate-poly (ethylene glycol) methacrylate block copolymer using RAFT block synthesis.

To a 50-ml air free flask were added glycidyl methacrylate (9.63 g, 67.77 mmol), 2-cyanoprop-2-yl dithiobenzoate (0.30 g, 1.36 mmol), recrystallized Vazo-64 (AIBN, 22.3 mg, 0.136 mmol) and acetonitrile (15 ml). The reaction vessel was fitted with a magnetic stirrer, and nitrogen was bubbled through the solution for 30 minutes to remove any dissolved oxygen. The reaction flask was then heated to 60° C. for 12 hours. The reaction mixture was then added slowly to 200 ml of ethyl ether with good mechanical stirring. The polymer precipitated and was collected by vacuum filtration. The solid was placed in a vacuum oven at 30° C. overnight to remove the ether leaving 4.50 g of polymer (Macro CTA for next reaction). Next, 3.06 g (0.845 mmol) of dried polymer was placed in a 250-ml air free flask with 116 ml toluene and 43 ml acetonitrile. The polymer (Macro CTA) was dissolved with magnetic stirrer. Vazo-64 (AIBN, 13.9 mg, 0.084 mmol) and mPEG-methacrylate (500), (84.50 g, 168.90 mmol) were added to the flask and the flask was bubbled through the solution for 30 min to remove any dissolved oxygen. The reaction flask was then heated to 60° C. for 12 more hours. The reaction mixture was then added slowly to 500 ml of ethyl ether with good mechanical stirring. The block polymer precipitated and was collected by vacuum filtration. The solid was placed in a vacuum oven at 30° C. overnight to remove the ether.

This reaction is generally shown below:

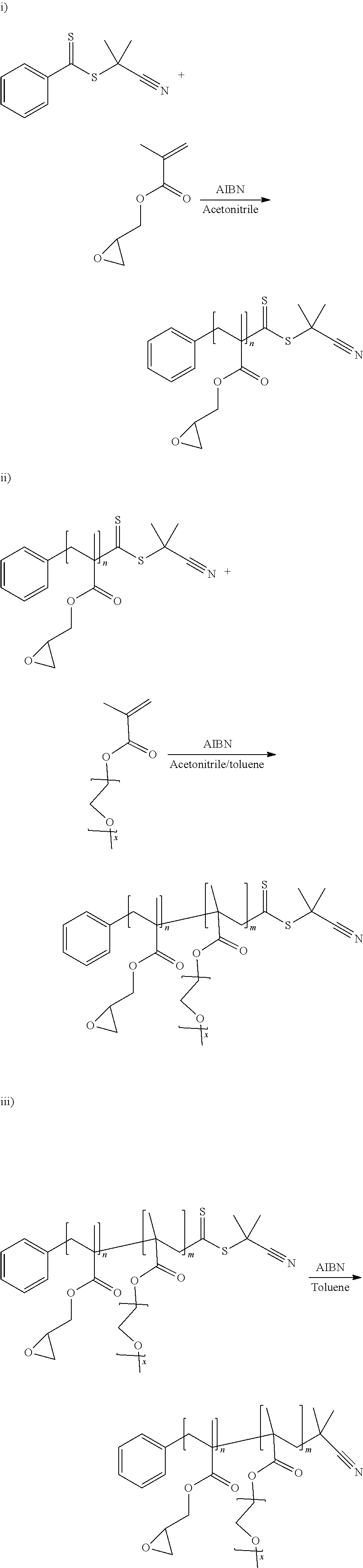

where m is 200, n is 15 and x is 500.

### EXAMPLE 3

Preparation of glycidyl methacrylate-methacryloyloxyethyl phosphorylcholine methacrylate block copolymer using ATRP block synthesis.

The glycidylmethacrylate-methacryloyloxyethylphosphorylcholine methacrylate block copolymer was prepared in a similar manner as Examples 1A and 1B. Methacryloyloxyethyl phosphorylcholine methacrylate (8.86 g, 30 mmol) was added to polyglycidyl methacrylate (0.161 g, 0.10 mmol) as made in Examples 1A and 1B, along with 30 ml of ethanol. The solution was purged for 1 hr under nitrogen. The reaction vessel was sealed and placed in a 60° C. oil bath for 8 hours. The reaction solution was concentrated and extracted with acetonitrile three times. The residue was dissolved in ethanol and passed through an aluminum oxide column. The polymer solution was precipitated in ethyl ether and dried in vacuum oven at 30° C. overnight to remove the ether.

### EXAMPLE 4

Preparation of glycidyl methacrylate-dimethylacrylamide block copolymer using RAFT block synthesis.

To a 50-ml air free flask was added distilled N,N-dimethylacrylamide (DMA, 9.9 g, 0.1 moles), 4-Cyano-4-[(dodecylsulfanylthiocarbonyl)sulfanyl]pentanoic acid (0.081 g, 0.2 mmol), recrystallized Vazo-64 (AIBN, 6.0 Mg, 0.36 Mmol) and toluene (25 ml). The reaction vessel was fitted with a magnetic stirrer, and nitrogen was bubbled through the solution for 30 minutes to remove any dissolved oxygen. The reaction flask was then heated to 60° C. for 12 hours. The reaction mixture was then added slowly to 200 ml of ethyl ether with good mechanical stirring. The polymer precipitated and was collected by vacuum filtration.

The solid was placed in a vacuum oven at 30° C. overnight to remove the ether leaving 9.35 g of polymer (Macro CTA for next reaction). The dried polymer was placed in a 50-ml air free flask with 25 ml toluene with magnetic stirrer to dissolve the polymer (Macro CTA) completely. Vazo-64 (AIBN, 6.0 mg, 0. 36 mmol) and glycidyl methacrylate (GMA, 0.46 g, 3.2 mmol) were added to the flask and the flask was bubbled through the solution for 30 minutes to remove any dissolved oxygen. The reaction flask was then heated to 60° C. for 12 more hours. The reaction mixture was then added slowly to 200 ml of ethyl ether with good mechanical stirring. The block polymer precipitated and was collected by vacuum filtration. The solid was placed in a vacuum oven at 30° C. overnight to remove the ether.

### EXAMPLES 5-8 AND COMPARATIVE EXAMPLE 1

Soflens Daily Disposal (SDD) contact lenses were used in this example. The contact lens is primarily comprised of poly 2-hydroxymethacrylate and poly n-vinyl pyrrolidone, ethylene glycol dimethacrylate, and allyl methacrylate.

The SDD contact lens was coated with the block copolymer of Example 1a. Each lens was placed in a polypropylene blister package with the packaging solution set forth in Table 1 below at amounts per weight percent, based on the total weight of the solution.

TABLE 1

| | | | | | Comp. |
|---|---|---|---|---|---|
| Formulation | Ex. 5 | Ex. 6 | Ex. 7 | Ex. 8 | Ex. 1 |
| Monobasic sodium phosphate | 0.015 | 0.015 | 0.015 | 0.015 | 0.015 |
| monohydrate | | | | | |
| Dibasic sodium phosphate | 0.065 | 0.065 | 0.065 | 0.065 | 0.065 |
| anhydrous | | | | | |
| Sodium Chloride | 0.900 | 0.900 | 0.900 | 0.900 | 0.900 |
| Purified Water, USP, q.s. | — | — | — | — | — |
| Block copolymer of Example 1a | 0.05 | 0.15 | 0.20 | 0.50 | — |

The lenses were then steam sterilized in the packaging solution.

To determine the success of the coating process, the contact angle, coefficient of friction, and fingertip lubricity were evaluated. It was determined that the lenses were highly lubricious when compared to the control lens (no coating) of Comparative Example 1.

The contact angle of the SDD contact lenses coated with the respective block copolymer of Examples 5, 7 and 8 in the phosphate buffered packaging solutions of Table 1 and the uncoated lens of Comparative Example 1 was evaluated. The results are set forth below in Table 2.

TABLE 2
SDD lenses - Contact Angle

| | Receding (°) | | Advancing (°) | | Sessile Drop | |
|---|---|---|---|---|---|---|
| Block Copolymer | Mean | SD | Mean | SD | Mean | SD |
| Ex. 8 (0.5 wt. %) | 37 | 1.8 | 42 | 5.2 | — | — |
| Ex. 7 (0.2 wt. %) | 36 | 1.1 | 43 | 3.9 | 76 | 4.0 |
| Ex. 5 (0.05 wt. %) | 36 | 1.0 | 41 | 3.1 | 65 | 1.5 |
| Comp. Ex. 1 (0 wt. %) | 34 | 0.2 | 47 | 2.4 | 77 | 2.4 |

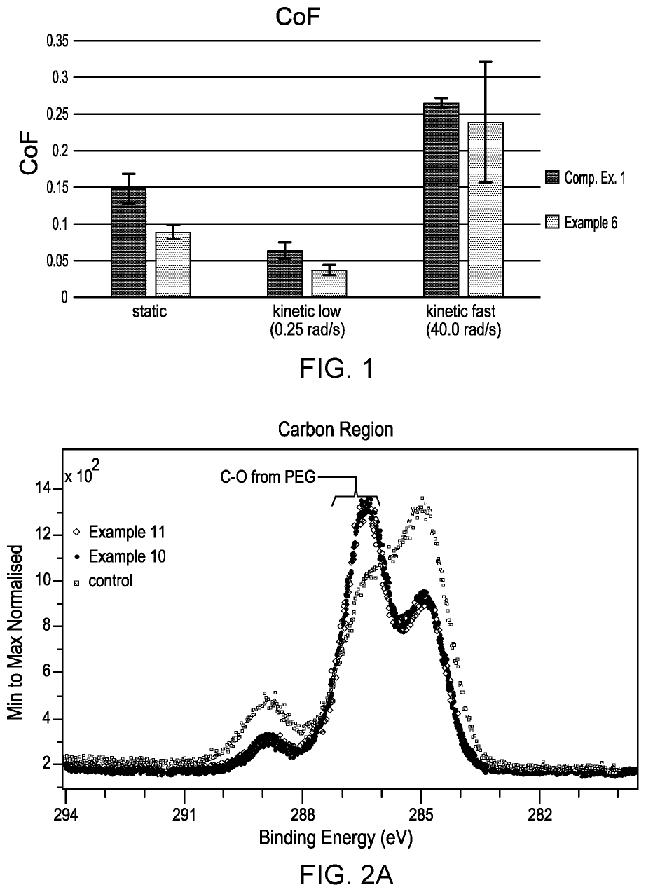

The coefficient of friction of the SDD contact lenses coated with the block copolymer of Example 6 in the phosphate buffered packaging solution of Table 1 and the uncoated lens of Comparative Example 1 was evaluated. As shown in FIG. 1, the low-speed kinetic coefficient of friction (0.25 rad/s) was decreased upon coating the lens.

### EXAMPLE 9 AND COMPARATIVE EXAMPLE 2

A representative SiHY contact lens was used in this example. The contact lens is primarily comprised of poly HEMA and poly DMA.

The SiHY contact lens was coated with the block copolymer of Example 4. The lens was placed in a polypropylene blister package with the packaging solution set forth in Table 3 below at amounts per weight percent, based on the total weight of the solution.

TABLE 3

| | | Comp. |
|---|---|---|
| Formulation | Ex. 9 | Ex. 2 |
| Monobasic sodium phosphate monohydrate | 0.015 | 0.015 |
| Dibasic sodium phosphate anhydrous | 0.065 | 0.065 |
| Sodium Chloride | 0.900 | 0.900 |
| Purified Water, USP, q.s. | — | — |
| Block copolymer of Example 4 | 0.30 | — |

The lenses were then steam sterilized in the packaging solution.

To determine success of the coating process, the Sudan black staining, sessile drop contact angle, and fingertip lubricity were evaluated. It was determined that the lenses were highly lubricious when compared to an uncoated SiHY contact lens of Comparative Example 2.

The Sudan black staining and sessile drop contact angle of the SiHY lens before and after sterilization in the packaging solution set forth in Table 3 was evaluated. The Sudan black binds to hydrophobic surfaces such that the less blue stain on the coated lens indicates a hydrophilic surface. When viewing the SiHY lens after the Sudan black staining was applied to the lens, it was seen that there was significantly less blue stain on the coated lens after sterilization thereby indicating a hydrophilic surface.

Also, the contact angle was lowered when the lens was coated. The results of the sessile drop data based on the contact angle measurement are set forth below in Table 4.

TABLE 4

| Comp. Ex./Ex. | Contact Angle | Average | Std. Dev |
|---|---|---|---|
| Comparative Ex. 2 | 95.18 | 93.8 | 0.95 |
| | 93.06 | | |
| | 93.28 | | |
| Example 9 | 89.97 | 89.7 | 0.55 |
| | 90.19 | | |
| | 88.92 | | |

### EXAMPLES 10 and 11 AND COMPARATIVE EXAMPLE 3

Methafilicon A lenses were used in this example. The contact lens is comprised of HEMA and Methacrylic Acid.

The Methafilicon A contact lens was coated with the block copolymer of Example 1A and 1B of different PEG lengths. The lens was placed in a polypropylene blister package set forth in Table 5 below.

TABLE 5

| | | | Comp. |
|---|---|---|---|
| Formulation | Ex. 10 | Ex. 11 | Ex. 3 |
| Sodium chloride | 0.800 | 0.800 | 0.800 |
| Sodium bicarbonate | 0.150 | 0.150 | 0.150 |
| Poloxamine 1107 | 0.009 | 0.009 | 0.009 |
| Purified Water, USP, q.s. | — | — | — |
| Block copolymer of Example 1A | 0.25 | — | — |
| Block copolymer of Example 1B | — | 0.25 | — |

The lenses were then steam sterilized in the block copolymer-containing packaging solution.

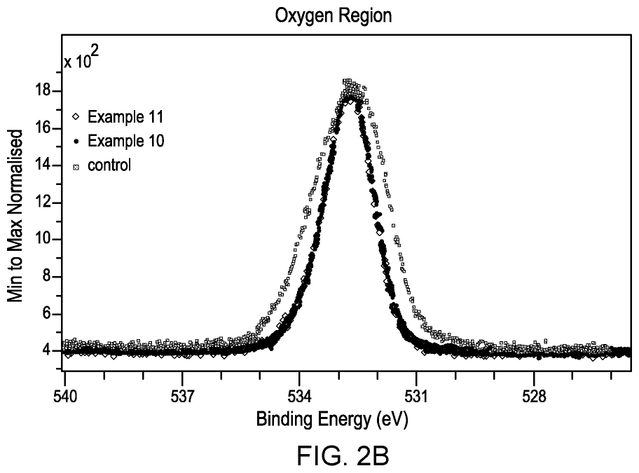

Next, XPS analysis below after washing the samples for 24 hours in a water bath showed a change in the chemical makeup of the surface of the lens. As can be seen in FIG. 2, there was a large increase in C—O bond in normalized spectra, indicating PEG on surface.

According to an aspect of the invention, a biomedical device having a surface coating comprises a bulk material having one or more biomedical device surface reactive functional groups and/or one or more biomedical device surface protected reactive functional groups and a surface coating, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

In one or more additional non-limiting illustrative embodiments, as may be combined with one or more of the preceding paragraphs, the surface coating comprises the block copolymer diffused in the bulk material.

In one or more additional non-limiting illustrative embodiments, as may be combined with one or more of the preceding paragraphs, the surface coating comprises the block copolymer adsorbed, entangled or covalently attached to the surface of the biomedical device through the one or more biomedical device surface reactive functional groups and/or the one or more biomedical device surface protected reactive functional groups.

In one or more additional non-limiting illustrative embodiments, as may be combined with one or more of the preceding paragraphs, the one or more biomedical device surface reactive functional groups of the biomedical device are selected from the group consisting of a hydroxy group, a tosylate group, a mesylate group, a triflate group, a nosyloxy group, an amino group, a carboxy group, a carbonyl group, an aldehyde group, a sulfonic acid group, a sulfonyl chloride group, an isocyanato group, a carboxy anhydride group, a lactone group, an azlactone group, an epoxy group, a group capable of undergoing Michael addition-type reaction and mixtures thereof.

In one or more additional non-limiting illustrative embodiments, as may be combined with one or more of the preceding paragraphs, the one or more biomedical device surface reactive functional groups of the biomedical device are selected from the group consisting of a hydroxy group, an amino group, a carboxy group and mixtures thereof and the ring-opening reactive functionalities that are complementary to the one or more biomedical device surface reactive functional groups are selected from the group consisting of an azlactone group, an epoxy group and mixtures thereof.

In one or more additional non-limiting illustrative embodiments, as may be combined with one or more of the preceding paragraphs, the ethylenically unsaturated containing monomer having ring-opening reactive functionalities contains 2 to about 18 carbon atoms which is substituted by a reactive group selected from the group consisting of an azlactone group, an epoxy group and mixtures thereof, and the ethylenically unsaturated-containing hydrophilic monomer contains 2 to about 18 carbon atoms which is substituted by a reactive group selected from the group consisting of an amide group, a carboxy anhydride group, a carboxy group, a hydroxy group and mixtures thereof.

In one or more additional non-limiting illustrative embodiments, as may be combined with one or more of the preceding paragraphs, the ethylenically unsaturated containing monomer having ring-opening reactive functionalities is selected from

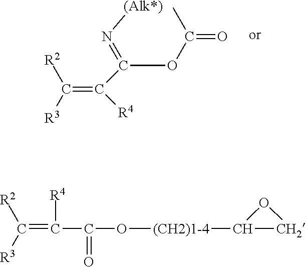

wherein R², R³ and R⁴ are each independently hydrogen or C₁-C₄ alkyl, and (Alk**) is a C₂-C₁₂ alkylene.

In one or more additional non-limiting illustrative embodiments, as may be combined with one or more of the preceding paragraphs, the ethylenically unsaturated-containing hydrophilic monomer is selected from the group consisting of an acrylamide, a formamide, a cyclic lactam, a (meth)acrylated alcohol, an ethylenically unsaturated polymerizable alkoxylated polymer, a methacrylated sulfobetaine, a methacrylated phosphocholine and mixtures thereof.

In one or more additional non-limiting illustrative embodiments, as may be combined with one or more of the preceding paragraphs, the ethylenically unsaturated containing monomer having ring-opening reactive functionalities are represented by the following structures:

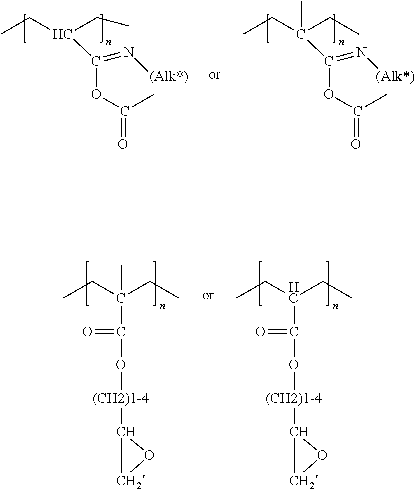

wherein n is from 1 to about 30; and wherein the monomeric units derived from an ethylenically unsaturated-containing hydrophilic monomer are monomeric units derived from an ethylenically unsaturated-containing hydrophilic monomer selected from the group consisting of an acrylamide, a formamide, a cyclic lactam, a (meth)acrylated alcohol, an ethylenically unsaturated polymerizable alkoxylated polymer, a methacrylated 1 sulfobetaine, a methacrylated phosphocholine and mixtures thereof.

In one or more additional non-limiting illustrative embodiments, as may be combined with one or more of the preceding paragraphs, the block copolymer comprises from about 10 to about 60 monomeric units derived from an ethylenically unsaturated containing monomers having ring-opening reactive functionalities and from about 70 to about 250 monomeric units derived from an ethylenically unsaturated-containing hydrophilic monomer.

In one or more additional non-limiting illustrative embodiments, as may be combined with one or more of the preceding paragraphs, the block copolymer comprises from about 15 to about 45 monomeric units derived from an ethylenically unsaturated containing monomers having ring-opening reactive functionalities and from about 100 to about 200 monomeric units derived from an ethylenically unsaturated-containing hydrophilic monomer.

In one or more additional non-limiting illustrative embodiments, as may be combined with one or more of the preceding paragraphs, the block copolymer comprises monomeric units derived from glycidyl methacrylate and monomeric units derived from poly(alkylene glycol).

In one or more additional non-limiting illustrative embodiments, as may be combined with one or more of the preceding paragraphs, the block copolymer is a brush block copolymer.

In one or more additional non-limiting illustrative embodiments, as may be combined with one or more of the preceding paragraphs, the block copolymer is a comb block copolymer.

In one or more additional non-limiting illustrative embodiments, as may be combined with one or more of the preceding paragraphs, the biomedical device is an ophthalmic lens.

In one or more additional non-limiting illustrative embodiments, as may be combined with one or more of the preceding paragraphs, the ophthalmic lens is a contact lens or an intraocular lens.

In one or more additional non-limiting illustrative embodiments, as may be combined with one or more of the preceding paragraphs, the biomedical device is a silicone contact lens or intraocular device.

In one or more additional non-limiting illustrative embodiments, as may be combined with one or more of the preceding paragraphs, the biomedical device is a silicone hydrogel continuous-wear lens.

According to another aspect of the invention, a method for making a biomedical device having a surface coating comprises forming a surface coating on a biomedical device having one or more biomedical device surface reactive functional groups and/or one or more biomedical device surface protected reactive functional groups, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

In one or more additional non-limiting illustrative embodiments, as may be combined with one or more of the preceding paragraphs, the step of forming the surface coating comprises diffusing the block copolymer in the biomedical device.

In one or more additional non-limiting illustrative embodiments, as may be combined with one or more of the preceding paragraphs, the step of diffusing the block copolymer in the biomedical device comprises (a) soaking the biomedical device in a swelling solution comprising one or more solvents and the block copolymer; and (b) removing the biomedical device from the swelling solution to provide the biomedical device comprising the block copolymer diffused in the biomedical device.

In one or more additional non-limiting illustrative embodiments, as may be combined with one or more of the preceding paragraphs, the step of forming the surface coating comprises exposing the biomedical device having one or more biomedical device surface reactive functional groups and/or one or more biomedical device surface protected reactive functional groups to the block copolymer comprising (a) monomeric units derived from an ethylenically unsaturated containing monomer having ring-opening reactive functionalities that are complementary to the one or more biomedical device surface reactive functional groups and (b) monomeric units derived from an ethylenically unsaturated-containing hydrophilic monomer to adsorb, entangle or covalently attach the ring-opening reactive functionalities of the monomeric units derived from an ethylenically unsaturated containing monomer having ring-opening reactive functionalities of the block copolymer to the one or more biomedical device surface reactive functional groups and the one or more biomedical device surface protected reactive functional groups of the biomedical device.

In one or more additional non-limiting illustrative embodiments, as may be combined with one or more of the preceding paragraphs, the one or more biomedical device surface reactive functional groups of the biomedical device are selected from the group consisting of a hydroxy group, a tosylate group, a mesylate group, a triflate group, a nosyloxy group, an amino group, a carboxy group, a carbonyl group, an aldehyde group, a sulfonic acid group, a sulfonyl chloride group, an isocyanato group, a carboxy anhydride group, a lactone group, an azlactone group, an epoxy group, a group capable of undergoing Michael addition-type reaction and mixtures thereof.

In one or more additional non-limiting illustrative embodiments, as may be combined with one or more of the preceding paragraphs, the one or more biomedical device surface reactive functional groups of the biomedical device are selected from the group consisting of a hydroxy group, amino group, carboxy group and mixtures thereof and the ring-opening reactive functionalities that are complementary to the one or more biomedical device surface reactive functional groups are selected from the group consisting of an azlactone group, an epoxy group and mixtures thereof.

In one or more additional non-limiting illustrative embodiments, as may be combined with one or more of the preceding paragraphs, the ethylenically unsaturated containing monomer having ring-opening reactive functionalities contains 2 to about 18 carbon atoms which is substituted by a reactive group selected from the group consisting of an azlactone group, an epoxy group and mixtures thereof, and the ethylenically unsaturated-containing hydrophilic monomer contains 2 to about 18 carbon atoms which is substituted by a reactive group selected from the group consisting of an amide group, a carboxy anhydride group, a carboxy group, a hydroxy group and mixtures thereof.

In one or more additional non-limiting illustrative embodiments, as may be combined with one or more of the preceding paragraphs, the ethylenically unsaturated containing monomer having ring-opening reactive functionalities is selected from

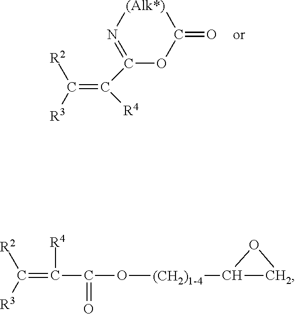

wherein R², R³ and R⁴ are each independently hydrogen or C₁-C₄ alkyl, and (Alk **) is a C₂-C₁₂ alkylene.

I n one or more additional non-limiting illustrative embodiments, as may be combined with one or more of the preceding paragraphs, the ethylenically unsaturated-containing hydrophilic monomer is selected from the group consisting of an acrylamide, a formamide, a cyclic lactam, a (meth)acrylated alcohol, an ethylenically unsaturated polymerizable alkoxylated polymer, a methacrylated sulfobetaine, a methacrylated phosphocholine and mixtures thereof.

In one or more additional non-limiting illustrative embodiments, as may be combined with one or more of the preceding paragraphs, the ethylenically unsaturated containing monomer having ring-opening reactive functionalities are represented by the following structures:

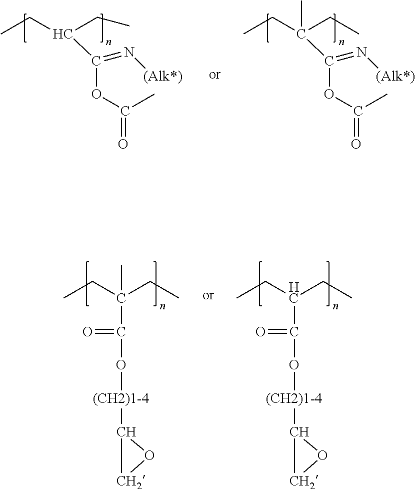

wherein n is from 1 to about 30; and wherein the monomeric units derived from an ethylenically unsaturated-containing hydrophilic monomer are monomeric units derived from an ethylenically unsaturated-containing hydrophilic monomer selected from the group consisting of an acrylamide, a formamide, a cyclic lactam, a (meth)acrylated alcohol, an ethylenically unsaturated polymerizable alkoxylated polymer, a methacrylated sulfobetaine, a methacrylated phosphocholine and mixtures thereof.

In one or more additional non-limiting illustrative embodiments, as may be combined with one or more of the preceding paragraphs, the block copolymer comprises from about 10 to about 60 monomeric units derived from an ethylenically unsaturated containing monomers having ring-opening reactive functionalities and from about 70 to about 250 monomeric units derived from an ethylenically unsaturated-containing hydrophilic monomer.

In one or more additional non-limiting illustrative embodiments, as may be combined with one or more of the preceding paragraphs, the block copolymer comprises from about 15 to about 45 monomeric units derived from an ethylenically unsaturated containing monomers having ring-opening reactive functionalities and from about 100 to about 200 monomeric units derived from an ethylenically unsaturated-containing hydrophilic monomer.

In one or more additional non-limiting illustrative embodiments, as may be combined with one or more of the preceding paragraphs, the block copolymer comprises monomeric units derived from glycidyl methacrylate and monomeric units derived from poly(alkylene glycol).

In one or more additional non-limiting illustrative embodiments, as may be combined with one or more of the preceding paragraphs, the block copolymer is a brush block copolymer.

In one or more additional non-limiting illustrative embodiments, as may be combined with one or more of the preceding paragraphs, the block copolymer is a comb block copolymer.

In one or more additional non-limiting illustrative embodiments, as may be combined with one or more of the preceding paragraphs, the biomedical device is an ophthalmic lens.

In one or more additional non-limiting illustrative embodiments, as may be combined with one or more of the preceding paragraphs, the ophthalmic lens is a contact lens or an intraocular lens.

In one or more additional non-limiting illustrative embodiments, as may be combined with one or more of the preceding paragraphs, the biomedical device is a silicone contact lens or intraocular device.

In one or more additional non-limiting illustrative embodiments, as may be combined with one or more of the preceding paragraphs, the biomedical device is a silicone hydrogel continuous-wear lens.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A biomedical device having a surface coating, comprising a bulk material having one or more biomedical device surface reactive functional groups and/or one or more biomedical device surface protected reactive functional groups and a surface coating, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

2. The biomedical device according to claim 1, wherein the surface coating comprises the block copolymer diffused in the bulk material.

3. The biomedical device according to claim 1, wherein the surface coating comprises the block copolymer adsorbed, entangled or covalently attached to the surface of the biomedical device through the one or more biomedical device surface reactive functional groups and/or the one or more biomedical device surface protected reactive functional groups.

4. The biomedical device according to claim 1, wherein the one or more biomedical device surface reactive functional groups of the biomedical device are selected from the group consisting of a hydroxy group, a tosylate group, a mesylate group, a triflate group, a nosyloxy group, an amino group, a carboxy group, a carbonyl group, an aldehyde group, a sulfonic acid group, a sulfonyl chloride group, an isocyanato group, a carboxy anhydride group, a lactone group, an azlactone group, an epoxy group, a group capable of undergoing Michael addition-type reaction and mixtures thereof.

5. The biomedical device according to claim 1, wherein the one or more biomedical device surface reactive functional groups of the biomedical device are selected from the group consisting of a hydroxy group, an amino group, a carboxy group and mixtures thereof and the ring-opening reactive functionalities that are complementary to the one or more biomedical device surface reactive functional groups are selected from the group consisting of an azlactone group, an epoxy group and mixtures thereof.

6. The biomedical device according to claim 1, wherein the ethylenically unsaturated containing monomer having ring-opening reactive functionalities contains 2 to about 18 carbon atoms which is substituted by a reactive group selected from the group consisting of an azlactone group, an epoxy group and mixtures thereof, and the ethylenically unsaturated-containing hydrophilic monomer contains 2 to about 18 carbon atoms which is substituted by a reactive group selected from the group consisting of an amide group, a carboxy anhydride group, a carboxy group, a hydroxy group and mixtures thereof.

7. The biomedical device according to claim 1, wherein the ethylenically unsaturated containing monomer having ring-opening reactive functionalities is selected from 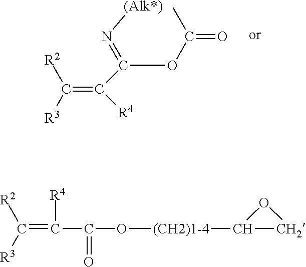
wherein R², R³ and R⁴ are each independently hydrogen or C₁-C₄ alkyl, and (Alk **) is a C₂-C₁₂ alkylene.

8. The biomedical device according to claim 1, wherein the ethylenically unsaturated-containing hydrophilic monomer is selected from the group consisting of an acrylamide, a formamide, a cyclic lactam, a (meth)acrylated alcohol, an ethylenically unsaturated polymerizable alkoxylated polymer, a methacrylated sulfobetaine, a methacrylated phosphocholine and mixtures thereof.

9. The biomedical device according to claim 1, wherein the ethylenically unsaturated containing monomer having ring-opening reactive functionalities are represented by the following structures: 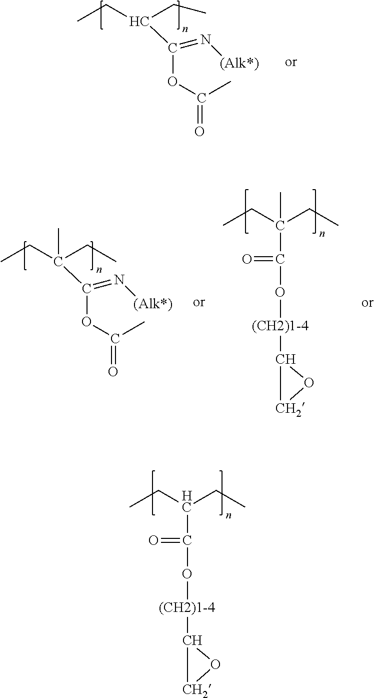
wherein n is from 1 to about 30; and wherein the monomeric units derived from an ethylenically unsaturated-containing hydrophilic monomer are monomeric units derived from an ethylenically unsaturated-containing hydrophilic monomer selected from the group consisting of an acrylamide, a formamide, a cyclic lactam, a (meth)acrylated alcohol, an ethylenically unsaturated polymerizable alkoxylated polymer, a methacrylated sulfobetaine, a methacrylated phosphocholine and mixtures thereof.

10. The biomedical device according to claim 1, wherein the block copolymer comprises from about 10 to about 60 monomeric units derived from the ethylenically unsaturated containing monomers having ring-opening reactive functionalities and from about 70 to about 250 monomeric units derived from the ethylenically unsaturated-containing hydrophilic monomer.

11. The biomedical device according to claim 1, wherein the block copolymer comprises from about 15 to about 45 monomeric units derived from the ethylenically unsaturated containing monomers having ring-opening reactive functionalities and from about 100 to about 200 monomeric units derived from the ethylenically unsaturated-containing hydrophilic monomer.

12. The biomedical device according to claim 1, wherein the block copolymer comprises monomeric units derived from glycidyl methacrylate and monomeric units derived from poly(alkylene glycol).

13. The biomedical device according to claim 1, wherein the block copolymer is one of a brush block copolymer or a comb block copolymer.

14. The biomedical device according to claim 1, wherein the biomedical device is an ophthalmic lens.

15. The biomedical device according to claim 14, wherein the ophthalmic lens is a contact lens or an intraocular lens.

16. The biomedical device according to claim 1, wherein the biomedical device is a silicone hydrogel continuous-wear lens.

17. A method for making a biomedical device having a surface coating, the method comprising forming a surface coating on a biomedical device having one or more biomedical device surface reactive functional groups and/or one or more biomedical device surface protected reactive functional groups, the surface coating being derived from a block copolymer comprising (a) monomeric units derived from an ethylenically unsaturated containing monomer having ring-opening reactive functionalities or nucleofugic functionalities that are complementary to the one or more biomedical device surface reactive functional groups and the one or more biomedical device surface protected reactive functional groups, and (b) monomeric units derived from an ethylenically unsaturated-containing hydrophilic monomer.

18. The method according to claim 17, wherein forming the surface coating comprises diffusing the block copolymer in the biomedical device.

19. The method according to claim 18, wherein diffusing the block copolymer in the biomedical device comprises (a) soaking the biomedical device in a swelling solution comprising one or more solvents and the block copolymer; and (b) removing the biomedical device from the swelling solution to provide the biomedical device comprising the block copolymer diffused in the biomedical device.

20. The method according to claim 17, wherein forming the surface coating comprises one of adsorbing, entangling or covalently attaching the block copolymer to the surface of the biomedical device through the one or more biomedical device surface reactive functional groups and/or the one or more biomedical device surface protected reactive functional groups.
